(19) United States
(12) Patent Application Publication
(10) Pub. No.: US 2023/0364303 A1
(43) Pub. Date: Nov. 16, 2023

(54) NERVE FASCICLE AND METHOD OF PRODUCING NERVE FASCICLE

(71) Applicant: University of Tsukuba, Tsukuba-shi, Ibaraki-ken, (JP)

(72) Inventors: Hiroshi ISHIKAWA, Tsukuba-shi, Ibaraki (JP); Aiki MARUSHIMA, Tsukuba-shi, Ibaraki (JP); Yuji MATSUMARU, Tsukuba-shi, Ibaraki (JP); Masao KODA, Tsukuba-shi, Ibaraki (JP); Akira MATSUMURA, Tsukuba-shi, Ibaraki (JP); Hiroki BUKAWA, Tsukuba-shi, Ibaraki (JP); Akihiro OHYAMA, Tsukuba-shi, Ibaraki (JP); Junko TOYOMURA, Tsukuba-shi, Ibaraki (JP); Miho WATANABE, Tsukuba-shi, Ibaraki (JP); Tetsuya ABE, Tsukuba-shi, Ibaraki (JP); Shohei TAKAOKA, Tsukuba-shi, Ibaraki (JP); Yosuke SHIBAO, Tsukuba-shi, Ibaraki (JP); Hideaki MATSUMURA, Tsukuba-shi, Ibaraki (JP)

(73) Assignee: University of Tsukuba, Tsukuba-shi, Ibaraki-ken, (JP)

(21) Appl. No.: 18/026,991

(22) PCT Filed: Sep. 17, 2021
(86) PCT No.: PCT/JP2021/034424

§ 371 Date: Mar. 17, 2023

(30) Foreign Application Priority Data
Sep. 18, 2020 (JP) 2020-157646

(51) Int. Cl. A61L 27/36; C12N 5/071; A61L 27/38

(52) U.S. Cl. CPC A61L 27/3675; C12N 5/0697; A61L 27/3804; A61L 2430/32; C12N 2502/086; C12N 2502/28; C12N 2502/1347; C12N 2502/1323; C12N 2501/10

(57) ABSTRACT

[Object] Provided is a method of producing a nerve fascicle including efficiently extending axons of neural cells.

[Solution] Neural cells are cultivated in the presence of feeder cells including at least one type of cells selected from the group consisting of vascular component cells, perivascular cells, and oligodendrocytes.

### TECHNICAL FIELD

The present invention relates to a nerve fascicle and a method of producing a nerve fascicle.

### BACKGROUND ART

In recent years, methods of transplanting nerves have been developed to restore the function of nerves that had been damaged due to neurodegenerative diseases and physical causes. As a method of transplanting nerves, widely used is a method of injecting neural stem cells into the affected part by injection. However, in this method, unfortunately, injected neural stem cells tend to move inside the body and are less likely to be engrafted, and thus it is difficult to efficiently repair the damaged site in neural tissues such as brain, spinal cord, complex peripheral nerves.

In order to solve this issue, methods have been developed for joining a bundle of neurons (nerve fascicle) with axons extended to the remaining nerves at the damaged site for transplanting. As a method for producing a nerve fascicle for transplantation, methods for cultivating neural cells so that axons of neurons extend so as to form a nerve fascicle have been reported (Patent Documents 1 and 2). In these methods, unfortunately, the axons of neurons cannot be efficiently extended and enlarged, and it is difficult to produce a nerve fascicle including axons having a length and a thickness (diameter) sufficient for transplantation.

In the case of central nervous system damage, particularly spinal cord injury, it is necessary to restore the nerve function within a short period after the damage from the viewpoint of the maintenance of the living body. Accordingly, it is necessary to form a nerve fascicle including axons having a length and a diameter sufficient for transplantation in a short period of time.

Under such circumstances, there is a need for a method in which a nerve fascicle including axons having a length and a diameter sufficient for transplantation can be efficiently produced.

### CITATION LIST

### Patent Literature

Patent Document 1: WO 2017/187696 A
Patent Document 2: JP 2014-136128 A

The present inventors have found that cultivation of neural cells in the presence of specific feeder cells and glial cells enables axons of neural cells to be efficiently extended. The present inventors have also found that the axons can be enlarged as axons of neural cells extend. Further, the present invention makes it possible to form a nerve fascicle including axons having a length and a diameter sufficient for transplantation efficiently. The present invention is based on these findings.

The present invention includes the following invention.
[1] A method of producing a nerve fascicle, the method including cultivating a neural cell-containing cell population in the presence of glial cells and at least one type of feeder cells selected from the group consisting of vascular component cells, perivascular cells, and oligodendrocytes to extend axons of neural cells.
[2] The method according to [1], in which the feeder cells include at least one type of cells selected from the group consisting of pericytes, vascular endothelial cells, fibroblasts, smooth muscle cells, and oligodendrocytes.
[3] The method according to [1] or [2], in which the feeder cells include cells secreting at least one type of growth factor selected from the group consisting of VEGF, NGF, BDNF, FGF-2, NGFB, and EGF.
[4] The method according to any one of [1] to [3], in which the glial cells include oligodendrocytes.
[5] The method according to any one of [1] to [4], in which the nerve fascicle includes a myelin sheath containing oligodendrocytes.
[6] The method according to any one of [1] to [5], including:
(a) preparing a substrate including at least one recess and a channel portion connected to the recess, the channel portion being covered with the feeder cells;
(b) adding the neural cell-containing cell population and the glial cells to the recess; and
(c) cultivating the neural cell-containing cell population and the glial cells to extend the axons of the neural cells along the channel portion.
[7] The method according to any one of [1] to [6], including:
(a) preparing a substrate including two recesses and a channel portion connecting the two recesses, the channel portion being covered with the feeder cells;
(b) adding the neural cell-containing cell population and the glial cells to each of the two recesses; and
(c) cultivating the neural cell-containing cell population and the glial cells to extend the axons of the neural cells along the channel portion.
[8] The method according to [6] or [7], in which, in the step (a), the channel portion is covered with fibroblasts before covered with the feeder cells.
[9] The method according to any one of [1] to [8], in which the neural cell-containing cell population further includes endothelial cells.
[10] The method according to [9], in which the endothelial cells included in the neural cell-containing cell population are blood vessel-derived endothelial cells.
[11] The method according to [10], in which the blood vessel-derived endothelial cells are blood vessel-derived endothelial cells in at least one tissue selected from the group consisting of dental pulp, a gingiva, a subcutaneous tissue, a coelomic artery, a coelomic vein, and an umbilical cord.
[12] The method according to any one of [9] to [11], further including, in the step (c), forming a tube derived from endothelial cells in the neural cell-containing cell population.
[13] The method according to any one of [9] to [12], in which the neural cells and the endothelial cells are derived from an identical individual.
[14] The method according to any one of [9] to [13], in which the neural cell-containing cell population further includes a biocompatible material, and the neural cells and the endothelial cells are layered on a surface of a different biocompatible material, respectively.
[15] The method according to [14], in which the biocompatible material includes a collagen.
[16] The method according to [14] or [15], in which the biocompatible material includes collagen beads.
[17] The method according to any one of [6] to [16], in which the channel portion has a length of 3 mm or greater.
[18] A nerve fascicle produced by the method described in any one of [1] to [17].
[19] A method of producing an implant including covering a nerve fascicle with a sheet of a biocompatible material, the nerve fascicle being produced by the method described in any one of [1] to [17].
[20] The method according to [19], in which the sheet includes fibroblasts.
[21] The method according to [19] or [20], in which the implant is a nerve regeneration implant.
[22] An implant produced by the method described in any one of [19] to [21].
[23] A method of extending axons of neural cells, the method including cultivating neural cells in the presence of glial cells and feeder cells including at least one type of cells selected from the group consisting of vascular component cells, perivascular cells, and oligodendrocytes.
[24] The method according to [23], in which the feeder cells include at least one type of cells selected from the group consisting of pericytes, endothelial cells, fibroblasts, smooth muscle cells, and oligodendrocytes.
[25] The method according to [23] or [24], in which the glial cells include oligodendrocytes.
[26] A nerve fascicle including: neural cells with axons extended; and a tube of endothelial cells present along the axons, in which the nerve fascicle includes at least one type of cells of HNK-1 carbohydrate-expressing cells or p75NTR-expressing cells, and the axons have a myelin sheath containing oligodendrocytes.
[27] The nerve fascicle according to [26], including HNK-1 carbohydrate-expressing cells and p75NTR-expressing cells.
[28] The nerve fascicle according to [26] and [27], including at least one type of cells selected from the group consisting of NS200-expressing cells, peripherin-expressing cells, myelin basic protein-expressing cells, S100-expressing cells, MPZ-expressing cells, periaxin-expressing cells, CD31-expressing cells, and PDGFRβ-expressing cells.
[29] The nerve fascicle according to any one of [26] to [28], including: NS200-expressing cells; peripherin-expressing cells; myelin basic protein-expressing cells; S100-expressing cells; MPZ-expressing cells; periaxin-expressing cells; CD31-expressing cells; and PDGFRβ-expressing cells.
[30] The nerve fascicle according to any one of [26] to [29], including a cell layer including at least one cell selected from fibroblasts or pericytes that covers, along the axons of the neural cells, at least some of the axons, in which the tube of endothelial cells is present in the cell layer.
[31] The nerve fascicle according to any one of [26] to [30], in which the tube of endothelial cells further includes pericytes.
[32] An implant including the nerve fascicle described in any of [26] to [31].
[33] The implant according to [32], which is a nerve regeneration implant.
[34] A device for producing a nerve fascicle including at least one first recess and at least one first channel portion having at least a first end and a second end, in which the first channel portion includes at least one second channel portion having at least a first end and a second end and extending in parallel with the first channel portion, the first recess is connected to the first end of the first channel portion and the first end of the second channel portion.
[35] The device according to [34], in which glial cells and at least one type of feeder cells selected from the group consisting of vascular component cells, perivascular cells, and oligodendrocytes are arranged in at least one of the first channel portion or the second channel portion.
[36] The device according to [34] or [35], in which a second recess is connected to the second end of the first channel portion and the second end of the second channel portion.
[37] The device according to any one of [34] to [36], in which the first recess is a recess having an opening with a diameter of 3 to 10 mm, a bottom being horizontal or curved into a recessed shape, and a depth of 4 to 8 mm, and the first channel portion is a channel portion having a bottom being horizontal or curved into a recessed shape, an edge with a width of 4 to 6 mm, a length of 2 to 3 cm between the first end and the second end, and a depth of 4 to 6 mm.
[38] The device according to any one of [34] to [37], in which the second channel portion is a channel portion having a bottom being horizontal or curved into a recessed shape, an edge with a width of 2 to 2.5 mm, and a depth of 1.5 to 2 mm.
[39] The device according to any one of [36] to [38], in which the second recess is a recess having an opening with a diameter of 2 to 5 mm, a bottom being horizontal or curved into a recessed shape, and a depth of 3 to 6 mm.
[40] The device according to any one of [34] to [39], in which the first channel portion has three of the second channel portions.
[41] The device according to any one of [34] to [40], in which the device includes polydimethylsiloxane.

The present invention makes it possible to extend axons of neural cells efficiently. Also, the present invention makes it possible to enlarge the axons of neural cells efficiently. Further, the present invention makes it possible to form a nerve fascicle including axons having a length and diameter sufficient for transplantation efficiently. Furthermore, the present invention makes it possible for a nerve fascicle to be produced, the nerve fascicle including neural cells and a tube of endothelial cells present along the axons of the neural cells.

### BRIEF DESCRIPTION OF DRAWINGS

FIGS. 1A and 1B are phase contrast micrographs of neural cells having a two-dimensional meshwork formed by cultivating dental pulp-derived neural cells in a petri dish coated with ornithine and/or lysine to cause the cells to be differentiated and grown. Arrows in FIG. TA indicate small cells generated and attached to the soma of the neural cells. Arrows in FIG. 1B indicate small cells generated and attached to the axons of the neural cells.

FIG. 2A is a photograph of a nerve fascicle for spinal nerves formed using the small cells generated and attached to the soma of the neural cells, immunostained with an anti-Olig2 antibody. FIG. 2B is a photograph of a nerve fascicle for spinal nerves formed using the small cells generated and attached to the soma of the neural cells, immunostained with an anti-S100 antibody.

FIG. 3A is a photograph of a nerve fascicle for peripheral nerves formed using the small cells generated and attached to the axons of the neural cells, immunostained with an anti-Olig2 antibody. FIG. 3B is a photograph of a nerve fascicle for peripheral nerves formed using the small cells generated and attached to the axons of the neural cells, immunostained with an anti-S100 antibody.

FIG. 4 each show a schematic view of a device for forming a nerve fascicle. FIG. 4A is a top view of the device. FIG. 4B is a cross-sectional view (clover shape) of a channel portion of the device. FIG. 4C is a schematic view of the entire device and the channel portion when various cells are added to the device. An enlarged view of a part of the channel portion in FIG. 4C (one of the leaflets of the clover) is a schematic view when fibroblasts and various feeder cells are sequentially added.

FIG. 5 is a graph showing a relationship between the cultivation period and the length of nerve fibers (axons) in the case of using various cells as feeder cells.

FIG. 6A is a phase contrast micrograph showing the shape of a nerve fascicle in the presence of feeder cells (vascular pericytes). FIG. 6B is a phase contrast micrograph showing the shape of a nerve fascicle in the absence of feeder cells (vascular pericytes).

FIG. 7 show a macroscopic photograph and photographs of a nerve fascicle (intravascular nerve fascicle for the spinal cord) formed by a method of Example 7, immunostained. FIG. 7A is a macroscopic photograph. FIG. 7B is a photograph of the nerve fascicle, immunostained with an anti-vWF antibody and an anti-NF200 antibody. FIG. 7C is a photograph of the nerve fascicle, immunostained with an anti-vWF antibody and an anti-NF200 antibody.

FIG. 8 is a photograph showing a cross-section of the nerve fascicle, subjected to immunofluorescence staining with NF200 and myelin basic protein.

FIG. 9 is a photograph showing a cross-section of a nerve fascicle subjected to immunofluorescence staining with NF200 and von Willebrand factor.

FIG. 10A is a photograph in which only the T9 vertebral arch is excised in a rat. FIG. 10B is a photograph of an excised portion after excision of the T9 vertebral arch and thoracic cord of a rat.

FIG. 11A is a photograph of a transplantation target site immediately before transplantation of a nerve fascicle implant into the spinal cord (thoracic cord) of a rat. FIG. 11B is a photograph of a transplanted site immediately after transplantation of the nerve fascicle implant into the spinal cord (thoracic cord) of the rat.

FIG. 12 is a graph showing motor function recovery (BBB score) after transplantation (excision) in rats whose thoracic cord has been excised and rats which has been subjected to transplantation of a nerve fascicle implant after excision of the thoracic cord.

FIG. 13A is an image of hematoxylin-eosin (HE) staining of a vertical cross-section of an excised site 6 weeks after excision of the thoracic cord in a rat into which no nerve fascicle implant has been transplanted after the excision of the thoracic cord. FIG. 13B is an image of HE staining of a vertical cross-section of a transplanted site 6 weeks after implantation in a rat into which the nerve fascicle implant has been transplanted immediately after the excision of the thoracic cord.

FIG. 14A is an image of hematoxylin-eosin (HE) staining of a vertical cross-section of an excised and transplanted site 6 weeks after transplantation in a rat into which the nerve fascicle implant has been transplanted immediately after the excision of the thoracic cord. FIG. 14B is an enlarged HE stained image of a joint portion (portion indicated by an arrow in FIG. 14A) between the nerve fascicle implant and the nerve portion of the excised thoracic cord of the rat (host) in the stained image in FIG. 14A.

FIGS. 15A to 15C are photographs each showing a vertical cross-section of a nerve fascicle subjected to immunofluorescence staining with each of S100, p75NTR, and DAPI. FIG. 15D is a merged photograph of FIGS. 15A to 15C.

FIGS. 16A to 16D are photographs each showing a vertical cross-section of a nerve fascicle subjected to immunofluorescence staining with each of HNK-1 carbohydrate, p75NTR, MPZ, and DAPI. FIG. 16E is a merged photograph of FIGS. 16A to 16D.

FIG. 17 is a photograph showing a cross-section of a nerve fascicle subjected to immunofluorescence staining with NF200 and myelin basic protein.

FIG. 18A is a photograph showing a cross-section of a nerve fascicle subjected to immunofluorescence staining with NF200 and S100. FIG. 18B is a photograph showing a cross-section of a nerve fascicle subjected to immunofluorescence staining with NF200 and peripherin.

FIG. 19A is a photograph showing a cross-section of a nerve fascicle subjected to immunofluorescence staining with NF200 and periaxin. FIGS. 19B and 19C are photographs each showing a vertical cross-section of a nerve fascicle subjected to immunofluorescence staining with NF200 and periaxin.

FIGS. 20A and 20B are photographs each showing a cross-section of a nerve fascicle subjected to immunofluorescence staining with CD31 and PDGFRβ.

FIG. 21A is a photograph showing a cross-section of a nerve fascicle subjected to immunofluorescence staining with CD31 and PDGFRβ. FIG. 21B is a partially enlarged photograph of the immunofluorescence staining photograph of FIG. 21A.

FIG. 22 is a partially enlarged photograph of a cross-section of a nerve fascicle subjected to immunofluorescence staining with CD31 and PDGFRβ.

FIG. 23A is a photograph of a transplanted site 12 weeks after transplantation of an autologous nerve in a rat. FIG. 23B is a photograph of a transplanted site 12 weeks after transplantation of a nerve fascicle implant in a rat. FIG. 23C is a photograph of a transplanted site 12 weeks after transplantation of an artificial nerve in a rat. FIG. 23D is a photograph of a transplanted site 12 weeks after incision of a sciatic nerve of a rat.

FIG. 24A is a mathematical equation for calculating a sciatic functional index. FIG. 24B is a graph showing the sciatic functional index after transplantation (incision) in the transplanted rats and the sciatic nerve incised rats.

FIG. 25 is a graph showing wet weights of gastrocnemius muscles at distal ends of the transplanted sites in the transplanted rats and the sciatic nerve incised rats.

FIGS. 26A to 26D are photographs each showing a cross-section of a nerve fascicle subjected to immunofluorescence staining with each of STEM121, p75NTR, MPZ, and DAPI. FIG. 26E is a merged photograph of FIGS. 26A to 26D.

### DETAILED DESCRIPTION OF THE INVENTION

### Method of Producing Nerve Fascicle

According to one aspect of the present invention, provided is a method of producing a nerve fascicle (also referred to as “production method of the present invention” hereinafter). According to the production method of the present invention, it is possible to form a nerve fascicle including axons having a length and diameter sufficient for transplantation.

The method of producing a nerve fascicle includes cultivating a neural cell-containing cell population in the presence of glial cells (neuroglial cells) and feeder cells including at least one type of cells selected from the group consisting of vascular component cells, perivascular cells, and oligodendrocytes to extend axons of neural cells.

The term “neural cell-containing cell population” used herein means a population of cells containing neural cells as described below. The neural cell-containing cell population may include cells other than neural cells.

Examples of neural cells contained in the neural cell-containing population include not only cells constituting the nervous system, but also cells that may differentiate into the cells constituting the nervous system. Specific examples of neural cells to be used include neural stem cells, cells that may differentiate into neurons or glial cells, cells being differentiated into neurons or glial cells (e.g., immature neurons, immature glial cells, etc.), and differentiated mature neurons (e.g., mature neurons, mature glial cells, etc.). These neural cells may be commercially available neural cells, may be neural cells produced by isolating neural cells from the living body and preparing the cells, or may be neural cells differentiated and induced from pluripotent stem cells such as ES cells or iPS cells. The neural cells may also be cells (autologous cells) derived from an individual to be transplanted, or may be cells (allogeneic cells) derived from an individual other than the individual to be transplanted. In a preferred embodiment, the neural cell-containing population includes neural stem cells, cells that may differentiate into neurons, cells being differentiated into neurons, and/or mature neurons. The neural cells included in the neural cell-containing cell population are not particularly limited as long as the cells do not exhibit antigenicity against a subject of transplantation of the nerve fascicle of the present invention. Cells derived from any origin can be used. Examples of the origin of neural cells include an individual that is identical to the subject of transplantation of the nerve fascicle, an individual different from the subject of transplantation of the nerve fascicle, and an HLA (Human Leukocyte Antigen) homo donor. Examples of the origin of neural cells include cells in which immune rejection is suppressed through modification of a human leukocyte antigen (HLA) gene by a genome editing technique, e.g., universal donor cells.

The neural cell-containing cell population may be in any form as long as the effects of the present invention are achieved, and is, for example, in the form of being suspended in a culture medium (such as αMEM, a Dulbecco's modified Eagle medium (DMEM), a mixed medium of Dulbecco's modified Eagle medium/Ham's F-12 (DMEM/F12); Ham's 10, Ham's 12 and RPMI1640 media; or various types of neuron culture media), in the form of suspension cells, or the like.

The content of neural cells in the neural cell-containing cell population is not particularly limited as long as the effects of the present invention are achieved, and the number of neural cells is, for example, from 10³ to 10¹⁰, preferably 10⁴ to 10⁹, and more preferably 10³ to 10⁸.

Examples of cells other than neural cells included in the neural cell-containing cell population include endothelial cells and erythrocytes. Endothelial cells capable of forming blood vessels are preferably used as the endothelial cells, and specific examples thereof include vascular endothelial cells. The cells other than neural cells included in the neural cell-containing cell population are not particularly limited as long as the cells do not exhibit antigenicity against the subject of transplantation of the nerve fascicle of the present invention. Cells derived from any origin can be used. Examples of the origin of cells other than neural cells includes an individual that is identical to the subject of transplantation of the nerve fascicle, an individual different from the subject of transplantation of the nerve fascicle, and an HLA (Human Leukocyte Antigen) homo donor. Examples of the origin of neural cells include cells in which immune rejection is suppressed through modification of a human leukocyte antigen (HLA) gene by a genome editing technique, e.g., universal donor cells.

In a case where the neural cell-containing cell population includes endothelial cells, the endothelial cells are expanded and differentiated by cultivating the neural cell-containing cell population in the presence of feeder cells, whereby a tube derived from endothelial cells is formed along the extended axons. As a result, the nerve fascicle thus produced has a tube of endothelial cells (blood vessel) that extends in the same direction as nerve fibers (axons) and adheres to nerve fibers (axons), in addition to a bundle of neural cells (nerve fibers) with extended axons.

Any of the endothelial stem cells, the cells being differentiated into the endothelial cells, and the differentiated mature endothelial cells can be used as the endothelial cells included in the neural cell-containing cell population. Further, the origin of the endothelial cells included in the neural cell-containing cell population is not particularly limited. It is preferable to use blood vessel-derived endothelial cells, it is more preferable to use blood vessel-derived endothelial cells in dental pulp, gingiva, subcutaneous tissue, coelomic artery, coelomic vein, or umbilical cord, and it is even more preferable to use blood vessel-derived endothelial cells in dental pulp. These endothelial cells may be commercially available endothelial cells, or may be the endothelial cells differentiated and derived from pluripotent stem cells such as ES cells or iPS cells. Further, the endothelial cells may also be autologous cells derived from an individual to be transplanted, or may be allogeneic cells derived from an individual other than the individual to be transplanted.

The content of the endothelial cells in the neural cell-containing cell population is not particularly limited as long as the effects of the present invention are achieved, and the number of endothelial cells is, for example, from 2×10³ to 3×10¹⁰, preferably from 2×10⁴ to 3×10⁹, and more preferably from 2×10⁵ to 3×10⁸. In a preferred embodiment, the number of the endothelial cells in the neuron-containing population is greater than the number of neural cells.

The neural cell-containing cell population may include a biocompatible material in addition to the aforementioned cells. The biocompatible material may be used without any particular limitation as long as the cells in the neural cell-containing cell population can adhere to the biocompatible material, and the cells in the process of cell growth and proliferation can metabolize and consume the biocompatible material. Examples of such biocompatible materials include collagen, laminin, fibronectin, gelatin, and Matrigel (trade name).

The shape of the biocompatible material is not particularly limited as long as the aforementioned effects are achieved. Examples of shapes of the biocompatible material include a bead (spherical or substantially spherical) shape, a rod shape, and a film shape.

Concerning the size of the biocompatible material with a bead shape, the diameter is, for example, from 50 to 400 μm, preferably from 100 to 300 μm, and more preferably from 100 to 200 μm.

The bead-shaped biocompatible material can be formed appropriately using a known method, and can be formed, for example, by adding a biocompatible material dropwise to an oil phase, so as to form droplets in the oil phase. Oils and fats in the oil phase are not particularly limited as long as droplets of the biocompatible material are formed. Examples of oils and fats include: edible oils such as corn oil, rapeseed oil, and sesame oil; and mineral oils derived from petroleum, natural gas, and coal (mineral oils). Further, the diameter of the beads (droplets) can be appropriately adjusted by changing the dropping amount (speed).

Preferably, the neural cells included in the neural cell-containing cell population are layered on a surface of the biocompatible material. Specifically, the layering of neural cells on the surface of the biocompatible material results in a bundle of respective neural cells with axons extended after cultivation, and this is advantageous in that a large diameter (thick) nerve fascicle can be easily formed.

As the method of layering neural cells on the surface of the biocompatible material, a known method can be used. An example of the method includes a method of mixing a biocompatible material and a neural cell-containing cell population, putting the mixture into a container such as a non-adhesive petri dish, and subjecting the mixture to gyratory culture.

As for the amount of the neural cell-containing cell population per unit mass (g) of the biocompatible material, for example, the number of neural cells is from 200 to 1000, preferably from 300 to 800, and more preferably from 400 to 600. In the gyratory conditions, gyratory culture is performed, for example, at a speed of 30 to 60 rpm, preferably a speed of 35 to 55 rpm, more preferably a speed of 40 to 50 rpm.

When the neural cell-containing cell population includes endothelial cells, it is preferable that the endothelial cells included in the neural cell-containing cell population be layered on the surface of the biocompatible material. Specifically, the layering of endothelial cells on the surface of the biocompatible material results in the contact and fusion of a plurality of biocompatible materials with layers of endothelial cells derived from endothelial cells included in the neural cell-containing cell population in the cultivating process. As a result, it is possible to form a tube of endothelial cells which has a surface comprised of endothelial cells and includes the biocompatible material therein. Then, the endothelial cells constituting the tube surface uptake, metabolize, and consume the biocompatible material in the tube in the process of endothelial cell growth and proliferation, resulting in the formation of a hollow tube of endothelial cells. It is advantageous that the formed tube of endothelial cells can serve as a tube (blood vessel) that supplies oxygen, nutrients, and the like to the neural cells in the nerve fascicle to be formed, thereby preventing the necrosis of the nerve fascicle. The method of layering endothelial cells on the surface of the biocompatible material may be the same method as the aforementioned method of layering neural cells on the surface of the biocompatible material.

In order to achieve the advantages of layering neural cells and endothelial cells on the surface of the biocompatible material, it is preferable that the neural cells and the endothelial cells be layered on the surface of a different biocompatible material, respectively. Specifically, it is preferable that the neural cells be layered on a surface of a biocompatible material, and the endothelial cells be layered on a surface of another biocompatible material.

The biocompatible material may be mixed, in advance, with a substance that promotes the growth and proliferation of neural cells (neural cell growth factor), such as a nerve growth factor (NGF), a fibroblast growth factor-β(β-FGF), a vascular endothelial growth factor (VEGF), a hepatocyte growth factor (HGF), or atrophic factor. The biocompatible material includes a neural cell growth factor, so that it is possible to more efficiently promote the growth of neural cells present on the surface of the biocompatible material.

The proportion of the neural cell growth factor mixed with the biocompatible material can be appropriately set depending on the size of the nerve fascicle to be formed, and the proportion is, for example, from 1 to 1000 ng/mg, from 5 to 750 ng/mg, and from 10 to 500 ng/mg, relative to the total mass of the biocompatible material.

The neural cell growth factors may be used alone, or may be further mixed with ganglioside 3 (GD3) as a type of carbohydrate. The amount of GD3 mixed with the biocompatible material is not particularly limited, and is, for example, from 10 to 100 ng/mg, from 10 to 75 ng/mg, and from 10 to 50 ng/mg. In the case of adding the biocompatible material and the neural cell growth factors to a recess separately, the neural cell growth factors (a neural cell growth factor and GD3 when GD3 is used) are put into the recess and then the neural cells are put into the recess, thereby promoting the extension of the axons.

When the neural cell-containing cell population includes endothelial cells, the biocompatible material may be mixed, in advance, with a substance that promotes the growth and proliferation of endothelial cells (endothelial cell growth factor), such as a vascular endothelial growth factor (VEGF). The endothelial cell growth factor included in the biocompatible material can more efficiently promote the growth of endothelial cells present on the surface of the biocompatible material.

The proportion of the endothelial cell growth factor mixed with the biocompatible material can be appropriately set depending on the size of the nerve fascicle formed, and the proportion is, for example, from 10 to 300 ng/mg, from 20 to 200 ng/mg, and from 30 to 100 ng/mg, relative to the total mass of the biocompatible material.

When the biocompatible material and endothelial cells are separately added to the recess, the biocompatible material and endothelial cells are preferably added to the recess in such a manner that the endothelial cells adhere to the surface of the biocompatible material. The addition of the biocompatible material and endothelial cells in this manner can further promote the formation of blood vessels with lumens.

When the neural cell-containing cell population includes endothelial cells, the biocompatible material may be mixed with erythrocytes in advance. The biocompatible material includes erythrocytes so that erythrocytes can be present within a hollow tube of endothelial cells formed in the process of cultivating the neural cell-containing cell population. As a result, a complete hollow tube of endothelial cells can be formed. Then, in the formed nerve fascicle, oxygen is delivered to the neural cells by erythrocytes and the culture solution via the formed hollow tube of endothelial cells, so that it is possible to prevent the necrosis of the nerve fascicle.

The amount of erythrocytes mixed with the biocompatible material can be set as appropriate depending on the size of the nerve fascicle to be formed. The amount is, for example, from 2 to 20 mass %, preferably from 3 to 10 mass %, and more preferably from 4 to 7 mass %, relative to the amount of the biocompatible material.

The term “feeder cells” used herein refers to cells that produce and secrete substances that induce or retain the survival, proliferation, and differentiation of neural cells and optionally endothelial cells, and promotes the extension and enlargement of neural cells. The substances produced and secreted by the feeder cells are not particularly limited as long as the substances have the aforementioned functions. Examples of such substances include a nerve growth factor (NGF), a vascular endothelial growth factor (VEGF), a hepatocyte growth factor (HGF), a fibroblast growth factor (FGF-β), atrophic factor, a growth hormone-like substance, and IGF-1.

Feeder cells include cells that produce and secrete the substances as aforementioned. Specifically, the feeder cells include at least one type of cells selected from the group consisting of vascular component cells constituting blood vessels, perivascular cells around the blood vessels and oligodendrocytes.

Oligodendrocytes are one type of glial cells in the central nervous system and are also referred to as oligodendroglial cells. In a case where the glial cells as described below include oligodendrocytes, the feeder cells may include oligodendrocytes. Preferably, the feeder cells do not include oligodendrocytes.

Vascular component cells are not particularly limited as long as the effects of the present invention are achieved. Examples of vascular component cells include pericytes (e.g., vascular pericytes) and endothelial cells (e.g., vascular endothelial cells). Further, examples of perivascular cells include fibroblasts (e.g., perivascular fibroblasts) and smooth muscle cells (e.g., vascular smooth muscle cells).

The “glial cells” used herein refer to all cells that are not neurons among cells constituting the nervous system and assist the survival, development, and functional expression of neurons, and examples thereof include oligodendrocytes (oligodendroglial cells), astrocytes (astrocyte cells), Schwann cells (sheath cells), microglia (microglia cells), and ependymal cells. According to a preferred aspect of the present invention, the glial cells include oligodendrocytes.

According to a preferred embodiment of the present invention, the glial cells used in the present invention are prepared by the following method.

First, dental pulp from removed teeth are treated with an enzyme (trypsin, collagenase, Dispase, or the like) to separate cells constituting the dental pulp. Next, the separated cells are seeded on a hydrophilic petri dish and cultivated in a nerve induction medium (e.g., differentiation induction medium described in Takahashi et al. (Human Cell, Vol. 30, Issue 2, pp. 60-71) to which EGF and/or FGF are optionally added) for 1 to 2 days at a temperature of 37° C. and a CO₂ Concentration of 4.7 to 5% to form small spherical cells. Then, the small spherical cells are seeded on a non-cell adhesive petri dish and subjected to gyratory culture in a maintenance medium (e.g., B27 Plus Neuronal Culture System) at a temperature of 37° C. and a CO₂ concentration of 4.7 to 5% for about 1 day. Gyratory culture is performed preferably at a speed from 30 to 100 rpm, more preferably a speed from 40 to 80 rpm, still more preferably a speed from 50 to 60 rpm. A cell aggregate (spheroid) is formed. After that, the formed cell aggregate is treated with trypsin-EDTA, collagenase or Dispase to separate cells constituting the cell aggregate. Thereafter, the separated cells are seeded on a hydrophilic petri dish to form neural cells. Then, the resulting neural cells are cultivated in a petri dish or flask coated with ornithine and/or lysine, thereby allowing the neural cells to differentiate and grow into a two-dimensional meshwork. Small cells are generated and attached to the soma and axon parts of the neural cells differentiated and grown into a meshwork. All of these small cells are glial cells, and the small cells generated and attached to the soma part of the neural cells differentiate particularly into oligodendrocytes among the glial cells, and the small cells generated and attached to the axon part differentiate into Schwann cells. Such a method enables glial cells, in particular oligodendrocytes and Schwann cells, to be efficiently prepared, and this is a surprising fact that has been hitherto unknown in the related art. Here, the use of a known method such as immunostaining makes it possible to simply confirm the fact that the small cells generated and attached to the cell soma part of the neural cells are oligodendrocytes or Schwann cells.

According to one embodiment, in a nerve fascicle produced by the method of the present invention, at least some, preferably all, of the neural cells in the nerve fascicle may have a myelin sheath including oligodendrocytes, which is a type of glial cells, in their axons. Thus, the nerve fascicle produced by the method of the present invention can be used, particularly as the nerve fascicle in the central nervous system such as brain and spinal cord. Specifically, the use of the nerve fascicle produced by the method of the present invention enables the motor function of four limbs or both lower limbs to be restored in a patient whose motor function of four limbs or both lower limbs is completely or partially lost due to severe spinal cord injury.

The method of producing a nerve fascicle is not particularly limited as long as the method includes a step of cultivating a neural cell-containing cell population in the presence of glial cells and feeder cells including at least one type of cells selected from the group consisting of vascular component cells, perivascular cells, and oligodendrocytes to extend axons of neural cells. The step can be performed, for example, as in steps (a) to (e) below.

### Step (a)

This step prepares a substrate including at least one recess and at least one channel portion connected to the recess, the channel portion being covered with feeder cells. In a preferred embodiment, the substrate includes at least one recess and a plurality of radially extending channel portions connected to the recess, the channel portions each having another recess (distal recess) at a distal end portion opposite to the recess. The number of channel portions is not particularly limited, and may be, for example, from 3 to 30, from 5 to 20, from 7 to 15, or the like. When the substrate includes a plurality of channel portions, a plurality of nerve fascicles can be formed at a time. In a preferred embodiment, endothelial cells are added to the recess of the substrate in advance before the recess is covered with the feeder cells. Specifically, about 10⁶ endothelial cells are added to the recess, the recess is left to stand still for 6 hours and then washed with Hanks' solution. Thus, the recess is covered with the endothelial cells.

The material of the substrate is not particularly limited as long as the effects of the present invention are achieved, and examples of materials of the substrate include dimethylpolysiloxane, polystyrene, and polypropylene.

The shape and dimensions of the substrate can be set as appropriate as long as the effects of the invention of the present application are achieved. The substrate preferably has at least one plane, and the shape is, for example, a rectangular parallelepiped, a cube, or a cylinder, and is preferably a rectangular parallelepiped.

The dimensions of the substrate are not particularly limited as long as the substrate includes at least one recess and a channel portion. For example, when the shape is a rectangular parallelepiped, the vertical and horizontal lengths of the rectangular parallelepiped substrate are from 5 to 20 cm, from 5 to 15 cm, from 5 to 10 cm, and the height (depth) of the substrate is from 1 to 10 cm, from 1 to 5 cm, and from 1 to 3 cm.

The substrate includes at least one recess for accommodating a neural cell-containing cell population and glial cells. When the substrate is provided with a plurality of recesses, the arrangement of the plurality of recesses is not particularly limited as long as the effects of the present invention are achieved. Preferably, the plurality of recesses is present on the same plane of the substrate. For example, the substrate includes two recesses, and the two recesses are present on the same plane of the substrate.

The shape and dimensions of the recess can be set as appropriate as long as the effects of the present invention are achieved. Examples of shapes of the recess include a cylindrical shape, a rectangular parallelepiped shape, a cubic shape, and a hemispherical shape.

When the shape of the recess is cylindrical, in the dimensions of the cylindrical recess, the diameter is, for example, from 1 to 15 mm, preferably from 1 to 10 mm, and more preferably from 1 to 7 mm, and the height (depth) is from 2 to 8 mm, preferably from 3 to 7 mm, and more preferably from 4 to 6 mm. When the substrate includes a plurality of recesses, the shapes and/or dimensions of the recesses may be identical to or different from each other.

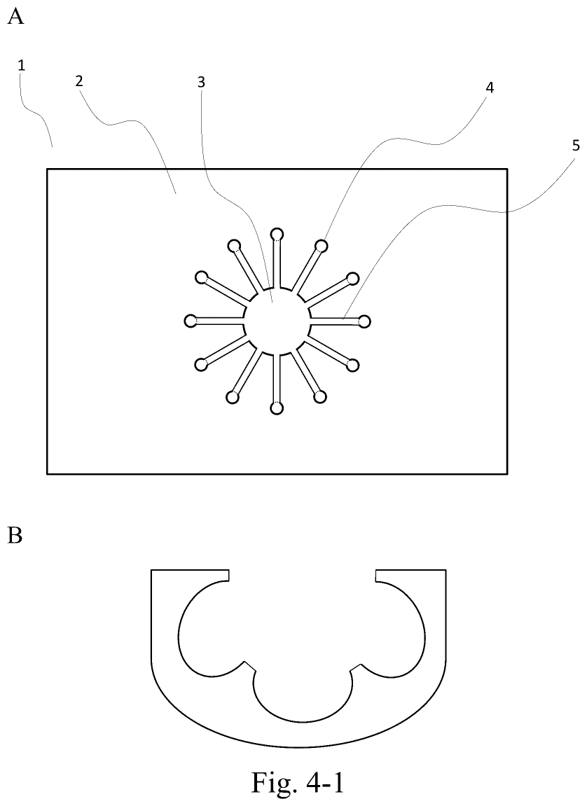

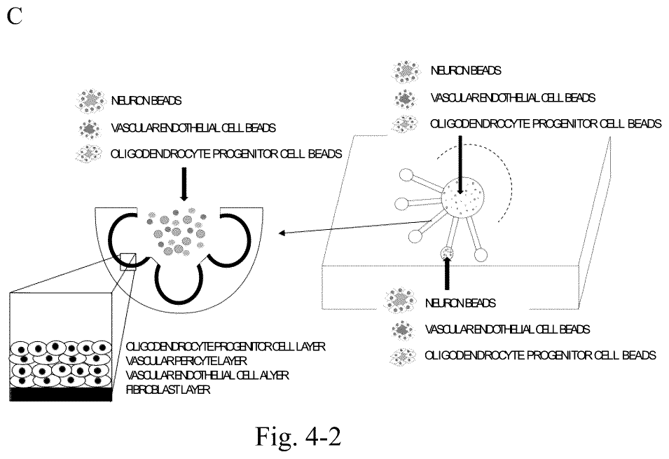

The substrate includes a channel portion connected to a recess. The channel portion may be covered with fibroblasts before covered with the feeder cells. In a case where the channel portion is covered with fibroblasts, the feeder cells are applied onto the fibroblast-covered channel portion. Further, the channel portion may also be covered with glial cells. In a case where the channel portion is covered with glial cells, the glial cells are preferably applied onto the feeder cells. In a preferred aspect of the present invention, the channel portion is first covered with fibroblasts, then endothelial cells as feeder cells are applied onto the fibroblast-covered channel portion. After that, pericytes as feeder cells are applied onto the resultant covered channel portion, and then glial cells are applied onto the pericyte-covered channel portion (see FIG. 4C).

The arrangement of the channel portion is not particularly limited as long as the effects of the present invention are achieved, and the channel portion and the recess are present on the same plane. Further, when the substrate includes a plurality of recesses, each of the recesses is connected to the channel portion, and the plurality of recesses is preferably connected through the channel portion. In a preferred embodiment, the substrate includes one central recess, a plurality of recesses radially present around the central recess, and channel portions connecting the central recess and the plurality of recesses radially present. The number of recesses radially present around the central recess is not particularly limited, and may be, for example, from 3 to 30, from 5 to 20, from 7 to 15, or the like. When the substrate has such a structure, a plurality of nerve fascicles or a thick nerve fascicle formed by bundling the nerve fascicles can be produced at a time.

The shape (cross-sectional shape) and the dimensions (length and depth) of the channel portion can be set as appropriate as long as the effects of the present invention are achieved. Examples of shapes of the channel portion include a clover shape, a concave shape, a V-shape, a U-shape, and an Ω-type shape. In a preferred embodiment, the channel portion has a clover shape. When the substrate includes a clover-shaped channel portion, the diameter of the cross section of each leaflet of the clover may be, for example, from 0.5 to 2 mm, from 0.75 to 1.5 mm, or from 1 to 1.2 mm.

The length of the channel portion (distance from the recess) is, for example, from 3 to 50 mm, preferably from 15 to 40 mm, and more preferably from 20 to 30 mm. The depth of the channel is, for example, from 1 to 10 mm, preferably from 2 to 7 mm, and more preferably from 3 to 5 mm. The width of the channel is, for example, from 1 to 5 mm, preferably from 1.5 to 4 mm, and more preferably from 2 to 3 mm. When the substrate includes a plurality of recesses, the length of the channel portion refers to a length (distance) between the recesses.

### Step (b)

In this step, the neural cell-containing cell population and glial cells are added to the recess. In a preferred embodiment, the neural cell-containing cell population and glial cells in a state of being layered on a different biocompatible material, respectively, preferably surfaces of beads such as collagen beads, is added to the recess. When the neural cell-containing cell population includes endothelial cells, the neural cells and the endothelial cells in the neural cell-containing cell population are added to the recess in a state in which the neural cells and the endothelial cells are layered on a surface of a different biocompatible material, respectively, preferably surfaces of beads such as collagen beads. The amount of the neural cell-containing cell population to be added to the recess is not particularly limited as long as the effects of the present invention are achieved, and the number of neural cells is, for example, from 10³ to 10¹⁰, preferably from 10⁴ to 10⁹, and more preferably from 10⁵ to 10⁸. As for the amount of endothelial cells to be added to the recess when the neural cell-containing cell population includes endothelial cells, the ratio of the number of neural cells to the number of endothelial cells (number of neural cells: number of endothelial cells) is, for example, from 1:1 to 1:10, from 1:2 to 1:7, and from 1:3 to 1:5.

The amount of glial cells to be added to the recess is not particularly limited as long as the effects of the present invention are achieved, and the number of glial cells is, for example, from 10⁴ to 10⁶, preferably from 5×10⁴ to 5×10⁵, and more preferably from 8×10⁴ to 10⁵. Further, the ratio of the number of glial cells to the number of neural cells contained in the neural cell-containing cell population (the number of glial cells: the number of neural cells) is, for example, from 1:3 to 1:100, from 1:5 to 1:50, or from 1:10 to 1:30.

### Step (c)

In this step, axons of neural cells are extended along the channel portion of the substrate by adding a culture solution to the recess and the channel portion of the substrate and cultivating the neural cell-containing cell population and glial cells added to the recess. In a preferred embodiment, the neural cell-containing cell population and the glial cells are incubated under reflux. As the axons of the neural cells are extended, the glial cells (oligodendrocytes) adhere to the axons of the neural cells to form a myelin sheath of oligodendrocytes.

The culture solution to be added is not particularly limited as long as it is a culture solution that is normally used in cultivating neural cells. Examples of culture solutions include: basal media such as neural cell culture solution, DMEM and RPMI1640 media, EMEM, Ham's 10 and Ham's 12 media; and a modified basal medium DMEM/F12. Various amino acids such as L-glutamine and L-alanine, additives such as fetal bovine serum (FBS), albumin, and the like may be added to these culture media. In the case of adding FBS to each culture medium, the concentration of FBS in each culture medium is, for example, from 5 to 20 mass %, from 7 to 15 mass %, and from 10 to 15 mass %. In the method of adding a culture solution, the culture solution may be added to each of the recess and the channel portion, or the culture solution may be supplied to each of the recess and the channel portion by immersing the substrate in a container that has filled with the culture solution in advance.

Culture conditions are not particularly limited as long as the cells contained in the neural cell-containing cell population and glial cells grow and proliferate. Examples of culture conditions include static culture at a temperature of 35 to 38° C. and a CO₂ concentration of 3 to 5% for a culture time of 20 to 24 hours. Depending on the degree of extension of the axons of neural cells, the static culture may be combined with the reflux culture. Reflux culture conditions can be set, for example, as follows: a temperature of 35 to 38° C.; a CO₂ concentration of 3 to 5%; a reflux rate of 1 to 10 ml/hour (preferably a reflux rate of 2 to 5 ml/hour); and a culture time of 72 hours to 60 days.

### Nerve Fascicle Produced by Production Method of Present Invention

According to one aspect of the present invention, provided is a nerve fascicle produced by the method described above. The nerve fascicle produced by the production method of the present invention is subjected to appropriate processing as necessary, and the processed nerve fascicle can be used as an implant, particularly a central nerve regeneration implant.

A plurality of nerve fascicles is combined as necessary, and the resultant fascicle can be used as a bundle of nerve fascicles. The bundle of nerve fascicles can be formed by rolling (covering) the plurality of nerve fascicles with a biocompatible material sheet prepared in advance. The biocompatible material sheet is not particularly limited as long as the effects of the present invention are achieved. Examples of biocompatible material sheets include a fibroblast sheet, a collagen fiber network sheet, and an elastic fiber network sheet.

According to one embodiment, a plurality of nerve fascicles is arranged on a fibroblast sheet prepared in advance, and the plurality of nerve fascicles is rolled into a roll (nori-maki; vinegared rice rolled in dried laver) with the fibroblast sheet to form a bundle of nerve fascicles. The thickness (diameter) of the bundle of nerve fascicles can be optionally changed according to the number of nerve fascicles arranged on the fibroblast sheet. As described above, when the nerve fascicles are rolled with the fibroblast sheet, suturing the ends of the fibroblast sheet to the nerve lesion site (nerve stump) to be transplanted enables the nerve fascicles to be easily transplanted.

According to another embodiment, a plurality of nerve fascicles aligned and arranged without gaps is attached to a surface of the previously formed fibroblast sheet, two fibroblast sheets to which the nerve fascicles are attached in such a manner are prepared, two biocompatible material sheets are stacked, and thus surfaces having the nerve fascicles attached thereon are in contact with each other and the directions of the nerve fascicles are aligned, resulting in formation of a bundle of nerve fascicles. The thickness of the bundle of nerve fascicles can be optionally changed according to the number of nerve fascicles attached to the surface of the fibroblast sheet. Further, according to still another embodiment, one fibroblast sheet having a nerve fascicle attached thereon is prepared, and at the same time, a tube filled with a cell mixture containing neural cell beads, endothelial cell beads, and glial cell beads at a mass ratio of approximately 3:1:1 is prepared. Then, the prepared fibroblast sheet and tube are arranged, and thus at least one end of the nerve fascicle on the surface of the sheet is in contact with the end of the tube. The nerve fascicle and tube are integrally rolled with the fibroblast sheet to form a cylindrical-shaped sheet, and thereafter the cell mixture with which the tube is filled is released into the cylindrical-shaped sheet, and thus the cylindrical-shaped sheet is filled with the cell mixture. Subsequently, the cylindrical fibroblast sheet filled with the cell mixture is subjected to static culture for 3 hours, and then incubated under reflux. The process described above causes the cell mixture to be differentiated and grown, as a result of which the nerve fascicle is further extended. Consequently, it is possible to produce a longer bundle of nerve fascicles. Here, the tube to be used is, for example, a glass tube. Further, the thickness (diameter) of the tube is not particularly limited, and can be appropriately set according to the target bundle of nerve fascicles.

The nerve fascicles can be used as an implant (nerve regeneration implant) as a substitute for damaged nerves. The subject of transplantation is not particularly limited. For example, the nerve fascicle can be transplanted into the central nervous system, the peripheral nervous system, or the like, and is preferably transplanted into the central nervous system.

Nerve fascicles can also be formed using cells (autologous cells) derived from an individual to be transplanted, and can also be formed using cells (allogeneic cells) derived from an individual other than the individual to be transplanted. Therefore, the formation of the nerve fascicles of the present invention using autologous cells makes it possible to transplant (autotransplant) the nerve fascicles to an individual from which the cells as the material of the nerve fascicles are derived, while causing little rejection.

### Method of Extending Axons of Neural Cells

According to one aspect of the invention, provided is a method of extending axons of neural cells (hereinafter, also referred to as “method of the present invention”). The method of the present invention allows axons of neural cells to efficiently extend. Further, the method of the present invention can efficiently increase the diameter of axons of nerves.

The method of the present invention includes cultivating a neural cell-containing cell population in the presence of glial cells and feeder cells including at least one type of cells selected from the group consisting of vascular component cells, perivascular cells, and oligodendrocytes to extend axons of neural cells.

In the method of the present invention, the neural cell-containing cell population, feeder cells, and glial cells can be the same as described in the aforementioned method of producing a nerve fascicle.

In the method of the present invention, the specific conditions of the step of cultivating a neural cell-containing cell population in the presence of glial cells and feeder cells including at least one type of cells selected from the group consisting of vascular component cells, perivascular cells, and oligodendrocytes to extend axons of neural cells can be the same as described in the method of producing a nerve fascicle.

### Nerve Fascicle

According to one aspect of the present invention, provided is a nerve fascicle (hereinafter, also referred to as “nerve fascicle of the present invention”) including: neural cells with axons extended; and a tube of endothelial cells present along the axons. The nerve fascicle includes at least one type of cells of HNK-1 carbohydrate-expressing cells or p75NTR-expressing cells. The axons have a myelin sheath containing oligodendrocytes.

The HNK-1 (human natural killer-1) carbohydrate, also referred to as CD57, has been found as a carbohydrate antigen expressed on human natural killer cells recognized by the monoclonal antibody HNK-1. The HNK-1 carbohydrate is known to be expressed in the nervous system of many vertebrates, including humans.

The p75NTR (p75 neurotrophin receptor) is also referred to as a low affinity neurotrophic factor receptor and is a receptor for a 75 kDa single-pass transmembrane neurotrophic factor receptor belonging to the tumor necrosis factor (TNF) receptor superfamily.

Both the HNK-1 carbohydrate and the p75NTR are known as markers for neural crest stem cells that are primarily expressed at the early stage of development in which neural crest cells are separated from the dorsal neural tube in vertebrates (particularly humans). Hence, the HNK-1 carbohydrate and the p75NTR are expressed in the immature nervous system of vertebrates in the embryonic stage, whereas both the HNK-1 carbohydrate and the p75NTR are very weakly expressed or not expressed in the nervous system of vertebrates after embryonic development. Therefore, both the HNK-1 carbohydrate and the p75NTR are very weakly expressed or not expressed in the mature nervous system of normal adult vertebrates.

In general, it is very difficult and substantially impossible to obtain a nerve fascicle from the immature nervous system of the embryo at the early stage of development, and it is known that there is no nerve fascicle that includes cells expressing HNK-1 carbohydrate and/or p75NTR. Consequently, the nerve fascicle of the present invention is said to be very specific in that it substantially expresses HNK-1 carbohydrate and/or p75NTR.

In a preferred embodiment, the nerve fascicle of the present invention includes HNK-1 carbohydrate-expressing cells and p75NTR-expressing cells. The HNK-1 carbohydrate-expressing cells and the p75NTR-expressing cells may be identical or different. In other words, both the HNK-1 carbohydrate and the p75NTR may be expressed in one cell, and the NHK-1 carbohydrate and the p75NTR may be expressed in a different cell, respectively.

Concerning the HNK-1 carbohydrate and the p75NTR, “HNK-1 carbohydrate-expressing cells” and “p75NTR-expressing cells” refer to cells that substantially express HNK-1 carbohydrate and cells that substantially express p75NTR, respectively. Here, the phrases “substantially express” and “substantially expressed”, and the like refer to a state in which gene transcription products or translation products are produced in cells. Specifically, the p75NTR is bound to a nerve growth factor (NGF), a brain-derived neurotrophic factor (BDNF), neurotrophin-3 (NT-3), neurotrophin-4/5 (NT-4/5), or the like to control the extension of axons of neurons. Further, the NHK-1 carbohydrate also has an increased expression during the stage of nerve development and is involved in neural circuit formation. Both the p75NTR and the HNK-1 carbohydrate are markers for neural crest stem cells and are known to be very weakly expressed or not expressed in the mature nervous system. Accordingly, it can be said that the nerve fascicle of the present invention in which these markers are expressed are different from the known nerve fascicles and implants produced by using normal peripheral nerves or neurons in the mature nervous system.

In the present invention, expressions of HNK-1 carbohydrate and p75NTR in cells can be confirmed by detecting expressions of the genes by immunostaining with an antibody against HNK-1 carbohydrate and an antibody against p75NTR.

In a preferred embodiment, HNK-1 carbohydrate-expressing cells and/or p75NTR-expressing cells are neural cells. In other words, in a preferred embodiment, the nerve fascicle of the present invention includes neural cells expressing HNK-1 carbohydrate and/or p75NTR.

In a preferred embodiment, the nerve fascicle of the present invention includes at least one type of cells selected from the group consisting of cells that express NS200 (NS200-expressing cells), cells that express peripherin (peripherin-expressing cells), cells that express myelin basic protein (myelin basic protein-expressing cells), cells that express S100 (S100-expressing cells), cells that express MPZ (MPZ-expressing cells), cells that express periaxin (periaxin-expressing cells), cells that express CD31 (CD31-expressing cells), and cells that express PDGFRβ(PDGFR-expressing cells). The cells expressing the above genes may be identical or different. In other words, this embodiment encompasses a case where two or more of the genes described above are expressed in one cell. For example, NS200-expressing cells may be cells that express only NS200 of the genes described above, and may be cells that express any of the genes described above in addition to NS200.

In a particularly preferred embodiment, the nerve fascicle of the present invention includes all the following cells: NS200-expressing cells, peripherin-expressing cells, myelin basic protein-expressing cells, S100-expressing cells, MPZ-expressing cells, periaxin-expressing cells, CD31-expressing cells, and PDGFR-expressing cells.

All the following genes are known as expression markers for nervous system: NF200, peripherin, myelin basic protein, S100, MPZ, and periaxin. Specifically, NF200 (Neurofilament 200) and peripherin are each known as a neuronal marker for myelinated nerves. Here, S100 is also known as a marker for Schwann cells. The myelin basic protein, S100, MPZ (Myelin protein zero) and periaxin are each known as a marker for myelin sheath.

Meanwhile, both the CD31 gene and the PDGFRβ gene are known as expression markers for vascular system. Specifically, the CD31 is known as a marker for vascular endothelial cells. The platelet-derived growth factor receptor β (PDGFRβ) is also known as a marker for vascular pericytes and fibroblasts.

The presence or absence of expressions of the above genes can be confirmed by the same method as in the method of confirming the presence or absence of expressions of HNK-1 carbohydrate and p75NTR as aforementioned.

The nerve fascicle of the present invention has a tube of endothelial cells that is present along the axons of the neural cells. At least some of the endothelial cells constituting the tube are preferably blood vessel-derived endothelial cells, more preferably blood vessel-derived endothelial cells in the dental pulp, gingiva, subcutaneous tissue, coelomic artery, coelomic vein, or umbilical cord, and still more preferably blood vessel-derived endothelial cells in the dental pulp. Further, the endothelial cells may also be autologous cells derived from an individual to be transplanted with the nerve fascicle, or may be allogeneic cells derived from an individual other than the individual to be transplanted. It is advantageous that the tube of endothelial cells has the advantage that can serve as a tube (blood vessel) that supplies oxygen, nutrients, and the like to the neural cells in the nerve fascicle, thereby preventing the necrosis of the nerve fascicle.

In a preferred embodiment, the tube of endothelial cells as aforementioned further includes pericytes. The tube of endothelial cells further includes pericytes, specifically the tube of endothelial cells is lined with pericytes, as a result of which the tube of endothelial cells is reinforced. In a case where the nerve fascicle of the present invention is responsible for the vascular role of endothelial cells, the reinforced tube of endothelial cells allows blood to flow stably into the tube of endothelial cells.

In a preferred embodiment, the nerve fascicle of the present invention has a cell layer including at least one type of cell selected from fibroblasts or pericytes that covers, along the axons of the neural cells, at least some of, or preferably all of, the axons. In a particularly preferred embodiment, the nerve fascicle of the present invention has the aforementioned tube of endothelial cells in the cell layer.

The nerve fascicle of the present invention can be used as an implant, particularly a nerve regeneration implant as a substitute for damaged nerves. The subject of transplantation of the nerve fascicle is not particularly limited. For example, the nerve fascicle can be transplanted in the central nervous system, the peripheral nervous system, or the like, and is preferably transplanted in the peripheral nervous system.

### Device for Producing Nerve Fascicle

According to one aspect of the present invention, provided is a device for producing a nerve fascicle (also referred to as “device of the present invention” hereinafter). The device according to the present invention is a device including at least one first recess and at least one first channel portion having at least a first end and a second end.

The first channel portion includes at least one second channel portion having at least a first end and a second end and extending in parallel with the first channel portion.

The first recess is connected to the first end of the first channel portion and the first end of the second channel portion.

In one embodiment, the first recess is a recess having an opening with a diameter of 3 to 10 mm, a bottom being horizontal or curved into a recessed shape, and a depth of 4 to 8 mm. In a preferred embodiment, the first recess is a recess having an opening with a diameter of 8 mm, a bottom being horizontal or curved into a recessed shape, and a depth of 6 mm.

In one embodiment, the first channel portion is a channel portion having a bottom being horizontal or curved into a recessed shape, an edge with a width of 4 to 6 mm, a length of 2 to 3 cm between the first end and the second end, and a depth of 4 to 6 mm. In a preferred embodiment, the first channel portion is a channel portion having a bottom being horizontal or curved into a recessed shape, an edge with a width of 5 mm, a length of 2 to 3 cm between the first end and the second end, and a depth of 6 mm.

In one embodiment, the second channel portion is a channel portion having a bottom being horizontal or curved into a recessed shape, an edge with a width of 2 to 2.5 mm, and a depth of 1.5 to 2 mm. In a particularly preferred embodiment, the second channel portion is a channel portion having a bottom being horizontal or curved into a recessed shape, an edge with a width of 2.5 mm, and a depth of 2 mm.

The first channel portion may include one second channel portion or a plurality of second channel portions. In a preferred embodiment, the first channel portion has three second channel portions. In this case, the arrangement of the second channel portions in the first channel portion is not particularly limited as long as the effects of the present invention are achieved, but the arrangement as shown in FIG. 4B is preferred.

The device of the present invention may have a second recess in addition to the first recess. The number of the second recesses may be one or plural. In a preferred embodiment, the second ends of the first channel portion and the second channel portion are connected to the second recess. When there is a plurality of second recesses, the second ends of different first and second channel portions are connected to the second recesses, respectively.

In one embodiment, the second recess is a recess having an opening with a diameter of 2 to 5 mm, a bottom being horizontal or curved into a recessed shape, and a depth of 3 to 6 mm. In a preferred embodiment, the second recess is a recess having an opening with a diameter of 3.5 mm, a bottom being horizontal or curved into a recessed shape, and a depth of 4 mm.

The first and/or second channel portion is preferably surface-treated to improve hydrophilicity. The hydrophilic treatment is not particularly limited as long as it does not exhibit cytotoxicity, and examples thereof include plasma treatment.

The size of the device of the present invention is not particularly limited as long as the device can accommodate the portions described above. For example, the device has a length of 4 to 10 cm, a width of 4 to 10 cm, and a thickness of 0.8 to 1.5 cm, and the portions are formed on a plane having the length and width described above. In a preferred embodiment, the size of the device of the present invention is 8 cm in length, 8 m in width, and 1 cm in thickness.

The material of the device of the present invention is not particularly limited as long as it does not exhibit cytotoxicity, and examples thereof include polydimethylsiloxane.

In one embodiment, neural cells, vascular endothelial cells, and oligodendrocytes are arranged in the first recess. As each of these cells, the same cells as described in the production method of the present invention can be used.

In one embodiment, glial cells and at least one type of feeder cells selected from the group consisting of vascular component cells, perivascular cells, and oligodendrocytes are arranged in at least one of the first channel portion or the second channel portion. For each of these cells, the same cells as described in the production method of the present invention can be used.

It possible to form a nerve fascicle using the device of the present invention according to the production method of the present invention described above.

### EXAMPLES

Hereinafter, the present invention will be specifically described with reference to examples, but the present invention is not limited to these examples.

### Example 1: Preparation of Glial Cells

Glial cells used in this example were prepared according to the following procedure.

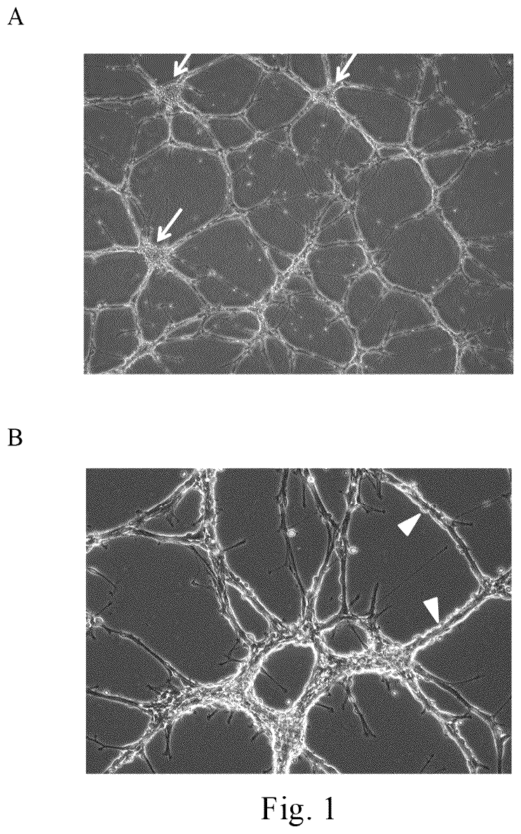

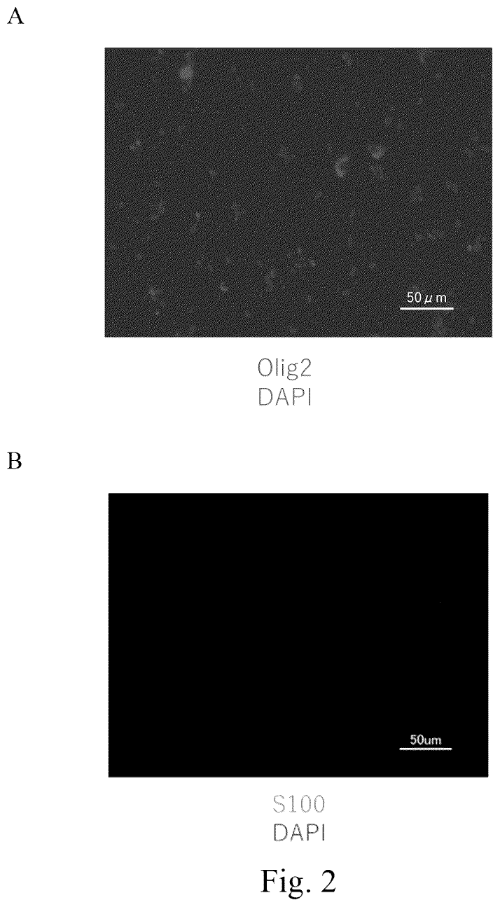

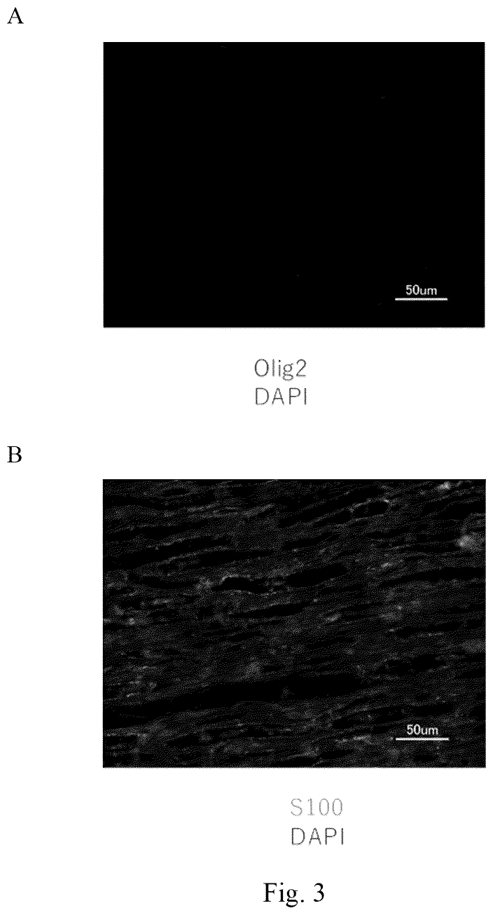

Dental pulp extracted from wisdom teeth (M3) were treated with an enzyme (0.1% trypsin and 0.02% EDTA in PBS (−)) to separate cells constituting the dental pulp. Next, the separated cells were seeded on a hydrophilic petri dish and cultivated in a nerve induction medium (differentiation induction medium described in Takahashi et al., Human Cell, Vol. 30, Issue 2, pp. 60-71) at a temperature of 37° C. and a CO₂ concentration of 4.7% for 1 to 2 days to be differentiated into small spherical cells. Then, the small spherical cells were collected by pipetting, the collected cells were centrifuged (1500 rpm) and seeded on a non-cell adhesive petri dish using a nerve maintenance medium, and subjected to gyratory culture (60 rpm) in the nerve maintenance medium at a temperature of 37° C. and a CO₂ concentration of 4.7% for about 1 day to form a cell aggregate (spheroid). After that, the formed cell aggregate was treated with trypsin-EDTA in PBS (−) to separate cells constituting the cell aggregate. Thereafter, the separated cells were seeded on a hydrophilic petri dish to form neural cells. Then, the resulting neural cells were cultivated in a nerve maintenance medium containing ornithine and lysine, and thus the neural cells were differentiated and grown into a two-dimensional meshwork. Phase contrast micrographs of the neural cells differentiated and grown into a two-dimensional meshwork are shown in FIGS. 1A and 1B. Subsequently, small cells generated and attached to each of the soma and axon parts of the neural cells differentiated and grown into a meshwork were collected (arrows in FIG. 1A and arrowheads in FIG. 1B indicate the small cells generated and attached to the soma part of the neural cells and the small cells generated and attached to the axon part of the neural cells, respectively). Then, the small cells were immunostained using an anti-S100 antibody and an anti-Olig2 antibody, and it was confirmed that the small cells generated and attached to the soma part of the neural cells were oligodendrocytes, one type of glial cells, and the small cells generated and attached to the axon part were Schwann cells, one type of glial cells. Hence, as shown in FIGS. 2A and 2B, the small cells generated and attached to the soma part were negative for S100 as one of the Schwann cell markers, whereas the small cells were positive for Olig2 as one of the oligodendrocyte markers. Meanwhile, as shown in FIGS. 3A and 3B, the small cells generated and attached to the axon part were positive for S100 as one of the Schwann cell markers, whereas the small cells were negative for Olig2 as one of the oligodendrocyte markers.

### Example 2: Formation of Neural Cell-Containing Cell Population

A neural cell-containing cell population used in this example was formed according to the following procedure.

Oral mesenchymal cells harvested from the human pulp, gingival epithelial tissue basal layer, and oral epithelial tissue basal layer were seeded onto a cell adhesive petri dish (manufactured by Falcon) and passaged till 3 to 7 passages at 37° C. and a CO₂ concentration of 4.5 to 5.5% using a mixed medium of Dulbecco's modified Eagle medium/Ham's F12 (DMEM/F12). The cells were separated with a trypsin-EDTA solution, the separated cells were seeded thinly onto a new petri dish, and large colonies with high growth potential were subjected to colonial cloning to obtain pulp-derived mesenchymal stem cells. The resultant stem cells were cultivated in a culture medium obtained by adding 10 ng/ml of epidermal growth factor (EGF) and 10 ng/ml of fibroblast growth factor-β(β-FGF) to a differentiation induction medium described in Takahashi et al. (Human Cell, Vol. 30, Issue 2, pp 60-71) at 37° C. and a CO₂ concentration of 4.5 to 5.5%, and differentiation induction was performed on the resultant culture to form a neural cell-containing cell population. Analyses by immunostaining and RT-PCR confirmed that neural stem cells, immature neurons, immature glial cells, mature neurons, and mature glial cells were included as the cells after differentiation induction. As a result of immunostaining of tyrosine hydroxylase, it was also confirmed that dopamine cells were included as the cells after differentiation induction.

### Example 3: Formation of Endothelial Cells

Endothelial cells used in this example were formed according to the following procedure.

The vascularized tissue harvested from the human oral cavity was treated with a digestive enzyme to separate the cells, and primary culture was performed on the separated cells. Colonies of morphologically identifiable endothelial cells were separated by colonial cloning, and the separated colonies were expanded to obtain endothelial cells.

### Example 4: Formation of Neural Cell Beads

Neural cell beads used in this example were formed according to the following procedure.

To an atelocollagen solution (manufactured by KOKEN CO., LTD.), a 10-fold concentrated Dulbecco's modified Eagle's medium/Ham's F-12 (DMEM/F12) mixed medium was added so as to be 10 mass %, and the resultant mixture was stirred and mixed to obtain an atelocollagen mixed liquid. A biocompatible material (collagen beads) was formed by adding the obtained atelocollagen mixed liquid dropwise to corn oil so that the diameter of particles (beads) of the atelocollagen was 200 μm. The obtained collagen beads were mixed with the neural cell-containing cell population formed in Example 2 at a mass ratio of 1:500. After mixing, the resultant mixture was put into a non-adhesive petri dish (available from IWAKI & CO., LTD.) and cultivated at a gyratory speed of 40 to 60 rpm, and the neural cell-containing cell population was layered on the surface of the collagen beads to form neural cell beads.

### Example 5: Formation of Endothelial Cell Beads

Endothelial cell beads used in this example were formed according to the following procedure.

To an atelocollagen solution (manufactured by KOKEN CO., LTD.), an 8- to 10-fold concentrated RPMI1640 medium was added so as to be 10 mass %. Further, a vascular endothelial growth factor (VEGF) (manufactured by Sigma-Aldrich Co. LLC) was added to be 50 ng/ml, erythrocytes were added to be 5 mass %, and the resultant mixture was stirred and mixed to obtain an atelocollagen mixed liquid. A biocompatible material (collagen beads) was formed by adding the obtained atelocollagen mixed liquid dropwise to corn oil so that the diameter of particles (beads) of the atelocollagen was 100 μm. The obtained collagen beads were mixed with the endothelial cells formed in Example 3 at a mass ratio of 1:500. After mixing, the resultant mixture was put into a non-adhesive petri dish (available from IWAKI & CO., LTD.) and cultivated at a gyratory speed of 40 to 60 rpm, and endothelial cells were layered on the surface of the collagen beads to form endothelial cell beads.

### Example 6: Production of Nerve Fascicle Forming Device

A device for forming a nerve fascicle was made according to the following procedure.

A cylindrical recess having a diameter of 5 mm and a depth of 5 mm was formed at the center on the surface of a dimethylpolysiloxane substrate (8 cm in length, 8 cm in width, and 1 cm in thickness). Further, twelve channel portions (3 cm in length, 4 mm in depth, and 2.5 mm in width) were formed radially around the recess. A cylindrical recess (distal recess, diameter: 3 mm, depth: 5 mm) was formed at the distal portion of each of the channel portions (distal portion on the side opposite to the side connected to the recess). A clover-shaped channel portion having leaflets with a diameter of about 1 mm is formed in each of the channel portions, and thus a device for forming a nerve fascicle is formed. A schematic view of the formed device and a schematic view of the clover-shaped channel portion are shown in FIGS. 4A and 4B, respectively.

### Example 7: Formation of Nerve Fascicles

Prior to the formation of nerve fascicles, feeder cells suitable for extending axons of neural cells were investigated by the procedure below.

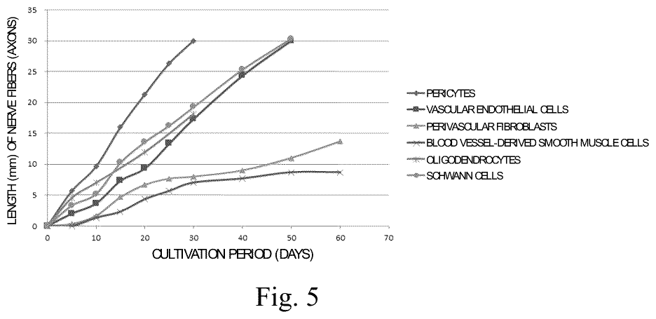

Four devices formed in Example 6 were prepared, and vascular component cells (vascular pericytes, vascular endothelial cells, and blood vessel-derived smooth muscle cells), perivascular cells (perivascular fibroblasts), oligodendrocytes, and Schwann cells as feeder cells were seeded onto each channel portion of each of the devices. Thereafter, each of the devices was placed in a culture vessel, and DMEM/F12 supplemented with 15 mass % fetal bovine serum (FBS) was added to the culture vessel. The culture vessel was left to stand still in a CO₂ incubator (at a temperature 37° C. and a CO₂ concentration of 4.7%), and the cells in each channel portion of each of the devices were cultivated to form a monolayer of each of the cells in each channel portion. Then, the neural cell beads formed in Example 4 and the endothelial cell beads formed in Example 5 were mixed at a mass ratio of 3:1, and the mixture was added to each of two recesses of each of the devices using a micropipette. The DMEM/F12 medium was added to each of the recesses, and each of the devices was left to stand still in a CO₂ incubator (at a temperature 37° C. and a CO₂ concentration of 4.7%) for cultivation for 20 hours. Each of the devices was then placed in a chamber (6 cm in length×6 cm in width×2 cm in thickness) and incubated under reflux (at a flow rate of 2 ml/min, a temperature of 37° C., and a CO₂ concentration of 4.7%) for 72 to 168 hours to cause axons of neural cells to extend on each of the cell layers in each channel portion. Then, a relationship between the cultivation period and the length of the nerve fascicles was investigated. The results are shown in FIG. 5. Although not shown in FIG. 5, when cultivation was performed using vascular pericytes, vascular endothelial cells, perivascular fibroblasts, and blood vessel-derived smooth muscle cells as feeder cells, on day 30 after the start of cultivation, the diameter of axons in each case were approximately from 100 to 150 μm, approximately from 50 to 80 μm, approximately from 20 to 30 μm, and approximately from 10 to 20 μm. Meanwhile, when feeder cells were not used, almost of the axons of neural cells were not enlarged, and the length on day 30 after the start of cultivation was approximately from 5 to 10 μm and the diameter was approximately from 5 to 10 μm.

The results of FIG. 5 showed that the axons of neural cells were efficiently extended when vascular component cells, perivascular cells, or oligodendrocytes were used as feeder cells. Particularly, it was shown that when vascular pericytes or vascular endothelial cells, i.e., vascular component cells, or oligodendrocytes were used as feeder cells, the axons of neural cells were extended very efficiently.

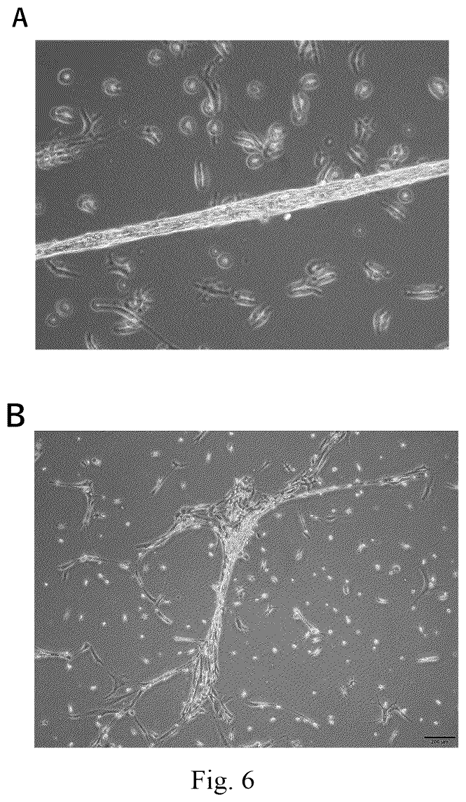

In addition, the influence of the presence or absence of feeder cells on the extension of the nerve fascicle in the case of forming the nerve fascicle using the device produced in Example 6 was preliminarily examined. Specifically, the extension of the nerve fascicle was observed in the presence or absence of vascular pericytes (perivascular fibroblasts) in each channel portion of the device. Phase contrast micrographs of the nerve fascicle in the presence or absence of vascular pericytes in the channel portion of the device are shown in FIGS. 6A and 6B, respectively. The photograph of FIG. 6A suggested that in the presence of vascular pericytes, oligodendrocytes were extended linearly, and the nerve fascicle was extended linearly. Meanwhile, the photograph of FIG. 6B suggested that in the absence of vascular pericytes, oligodendrocytes were extended in the form of branches, and the nerve fascicle was not extended linearly but extended in the form of branches. In general, when the nerve fascicle is used as the implant, a linear nerve fascicle is suitably used. Accordingly, it was suggested that feeder cells, particularly vascular pericytes, were preferably used to form the nerve fascicle.

A nerve fascicle was formed by the following procedure using vascular pericytes as feeder cells.

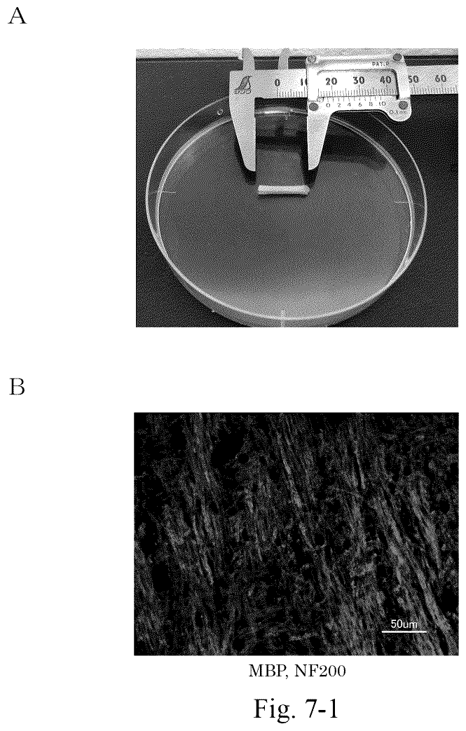

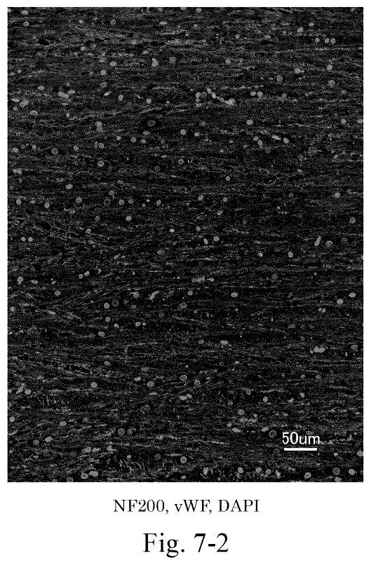

Fibroblasts were seeded onto each of the channel portions of the device formed in Example 6 to form a monolayer of fibroblasts. The human-derived vascular pericytes were then seeded onto fibroblasts to form a monolayer to multilayer of vascular pericytes. Thereafter, the device was placed in a culture vessel, and DMEM/F12 supplemented with 15 mass % fetal bovine serum (FBS) was added to the culture vessel. The culture vessel was left to stand still in a CO₂ incubator (at a temperature of 37° C. and a CO₂ concentration of 4.7%) to cultivate fibroblasts and vascular pericytes in each of the channel portions of the device, thereby forming a monolayer of fibroblasts and a monolayer of vascular pericytes adhered onto the fibroblast layer in each of the channel portions. Then, the glial cells (oligodendrocytes), the neural cell beads formed in Example 4 and the endothelial cell beads formed in Example 5 were mixed at a mass ratio of 1:4:2, and the mixture was added to each of the recesses of the device using a micropipette. The DMEM/F12 medium was added to each of the recesses, and the device was left to stand still in a CO₂ incubator (at a temperature 37° C. and a CO₂ concentration of 4.7%) for cultivation for 20 hours. Then, the device was placed in a chamber (6 cm in length×6 cm in width×2 cm in thickness) and incubated under reflux (at a flow rate of 2 ml/min, a temperature of 37° C., and a CO₂ concentration of 4.7%) for 30 days to cause axons of neural cells to extend on the fibroblast layer and the vascular pericyte layer in each of the channel portions, thereby obtaining a bundle of neural cells (nerve fibers) with axons extended (nerve fascicle). Here, the nerve fascicle thus obtained had a tube of endothelial cells (blood vessel) extended in the same direction as nerve fibers (axons) and adhered to nerve fibers (axons), in addition to the bundle of neural cells (nerve fibers) with axons extended. Further, the nerve fascicle had a length of about 3 cm and a diameter of about 1500 to 2200 μm. Furthermore, the fibroblasts constituting the fibroblast layer grew and proliferated to form a sheet of fibroblasts in each of the channel portions. FIG. 7 each show a micrograph (FIG. 7A) of the obtained nerve fascicle and photographs (FIGS. 7B and 7C) of immunostaining of the resulting nerve fascicle. FIG. 7B shows a myelin sheath of oligodendrocytes and axons of neural cells, and FIG. 7C shows a tube (blood vessel) of endothelial cells and axons of neural cells. From these results, it was confirmed that the nerve fascicle formed by the method of the present example had a tube of endothelial cells (blood vessel) present along axons and a myelin sheath of oligodendrocytes present along axons, in addition to the neural cells with axons extended.

The nerve fascicle formed as described above was subjected to immunofluorescence staining with NF200 and myelin basic protein to confirm the presence or absence of expressions of NF200 and myelin basic protein. Then, the nerve fascicle was cut at a right angle to the extension direction of axons of neurons. A photograph of the cut surface (cross-section) of the cut nerve fascicle is shown in FIG. 8. Expressions of NF200 and myelin basic protein around the NF200 were recognized from the photograph of FIG. 8. NF200 is known as a neuronal marker for myelinated nerves, and the myelin basic protein is known as a marker for oligodendrocytes. Thus, it was suggested from the photograph of FIG. 8 that the nerve fascicle included neurons with the myelin sheath of oligodendrocytes.

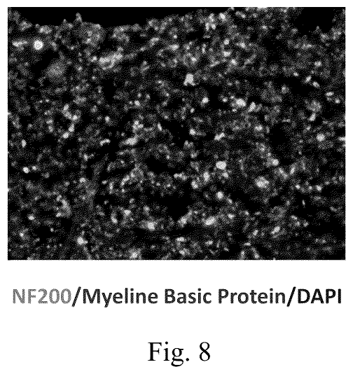

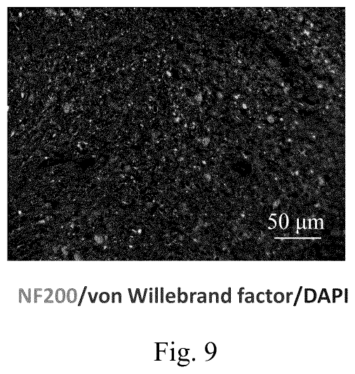

Similarly, the nerve fascicle was subjected to immunofluorescence staining with NF200 and von Willebrand factor to confirm the presence or absence of expressions of NF200 and von Willebrand factor. Then, the nerve fascicle was cut at a right angle to the extension direction of axons of neurons. Each photograph of the cut surface (cross-section) of the cut nerve fascicle is shown in FIG. 9. The expression of NF200 and circular expression of von Willebrand factor around the NF200 were observed from the photograph in FIG. 9. The NF200 is known as a neuronal marker for myelinated nerves and the von Willebrand factor is known as a marker for vascular endothelial cells. Accordingly, the photograph of FIG. 9 suggested that the nerve fascicle had neurons and a tube of vascular endothelial cells present along the axons of the neurons.

Next, the nerve fascicle formed in each channel portion was covered by rolling with the sheet of fibroblasts formed in the channel portion. Each of the nerve fascicles was then cut at the interface between the channel portion and each of the recesses at both ends and removed from the device. Six of the removed nerve fascicles were aligned and arranged without gaps on the surface of each of two large fibroblast sheets formed in advance in a petri dish, the nerve fascicles was left to stand still for 1 hour, and thus the nerve fascicles were attached to each of the fibroblast sheets. The two fibroblast sheets each having the six nerve fascicles attached thereon were stacked, and thus the surfaces with the nerve fascicles were in contact with each other and the directions of the nerve fascicles were aligned. The resultant sheet was left to stand still for 30 minutes. Then, the fibroblast sheet was cut along the long axis direction of the nerve fascicle to form an implant, and the implant was used in the transplantation as described below.

### Example 8: Transplantation of Nerve Fascicle 1 (Transplantation into Rat Spinal Cord)

The nerve fascicle implants formed in Example 7 was transplanted into spinal cord injury model rats according to the following method.

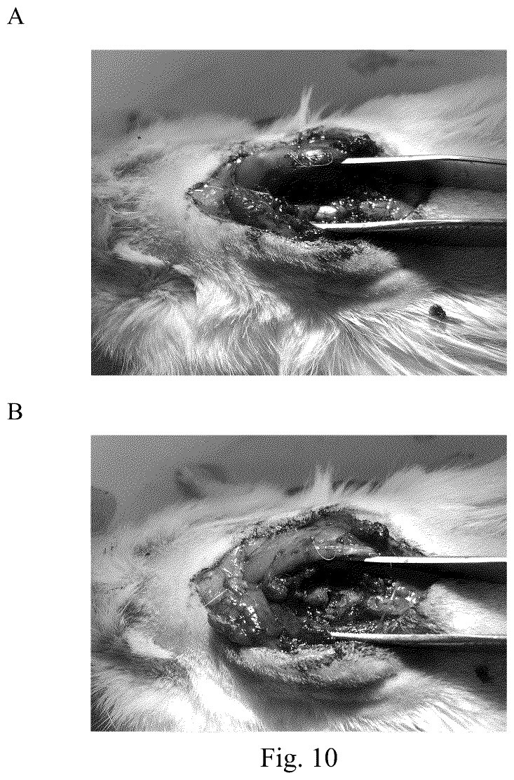

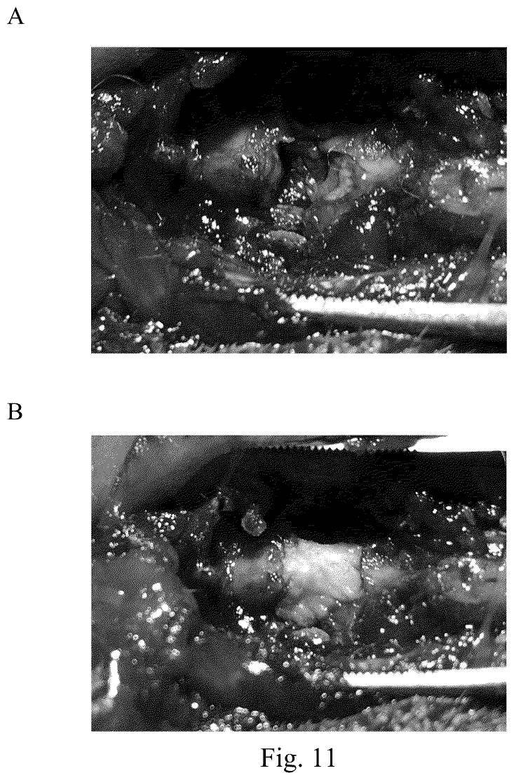

Rats (Wistar rat, 8-week-old, body weight about 200 g, available from Japan SLC, Inc.) were subjected to general anesthesia by intraperitoneal administration of three types of mixed anesthetic agents (a mixture of medetomidine hydrochloride (0.15 mg/0.15 ml/kg), midazolam (2 mg/0.4 ml/kg), butorphanol tartrate (2.5 mg/0.5 ml/kg), and physiological saline (1.45 ml/kg)), and the T9 vertebral arch and a part of the thoracic cord (2 mm as one segment of the thoracic cord) were excised. Each photograph of a rat after excision of the T9 vertebral arch and a part of the thoracic cord is shown in FIG. 10 (A: after the excision of the T9 vertebral arch, B: after the excision of the T9 vertebral arch and the thoracic cord). The nerve fascicle implant (length: about 5 to 8 mm, diameter: about 2 to 3 mm) formed in Example 7 was transplanted into the nerve portion of the excised thoracic cord. Each photograph of the transplanted site before and after transplantation of the nerve fascicle implant is shown in FIG. 11 (A: before the transplantation, B: after the transplantation). A white portion at the center of the photograph in FIG. 11B is the transplanted nerve fascicle implant. When the follow-up of the rats after transplantation of the nerve fascicle implant was observed, the rats became active similarly to autologous nerve-transplanted rats as controls and the movement of the left and right lower limbs was restored. Furthermore, there were no influences such as lifetime shortening.

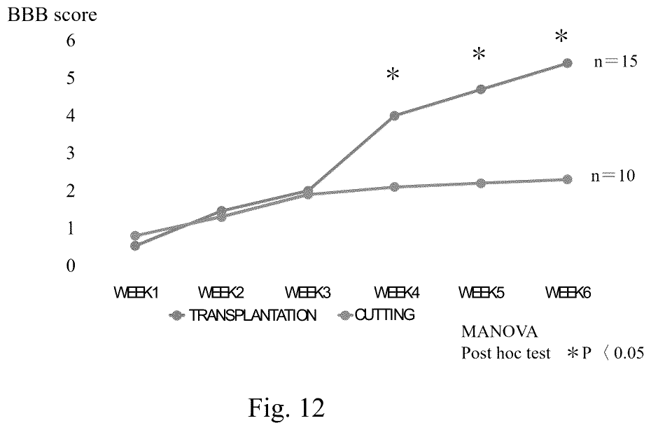

The motor function recovery in 10 thoracic cord-excised rats and 15 nerve fascicle implant-transplanted rats after the excision of the thoracic cord were evaluated based on Basso-Beattie-Bresnahan Locomotor Rating Scale (BBB score) as a commonly used indicator of the motor function recovery in spinal cord injury rats. The evaluation results are shown in FIG. 12. Weekly changes (1 to 6 weeks after transplantation) in BBB score in the group of nerve fascicle implant-transplanted rats were 0.5, 1.5, 2.0, 4.0, 4.7, and 5.4. Meanwhile, weekly changes in BBB score in the group of thoracic cord-excised rats were 0.8, 1.3, 1.9, 2.1, 2.2, and 2.3. As a result of the statistical study, in 4 weeks, 5 weeks, and 6 weeks after the transplantation, a significant difference was observed between the group of nerve fascicle implant-transplanted rats and the group of thoracic cord-excised rats. Further, in the group of nerve fascicle implant-transplanted rats, the motor function recovery of the lower limbs was observed 4 weeks after transplantation. Meanwhile, it was confirmed that in the group of nerve fascicle implant-transplanted rats, in a case where the transplanted sites in the rats 6 weeks after transplantation were excised again, the motor function of the lower limbs was deteriorated, and the BBB score was lowered to 1. From these results, it was found that the nerve fascicle implant-transplanted rats after the excision of the thoracic cord showed significantly higher motor function recovery, as compared with the thoracic cord-excised rats.

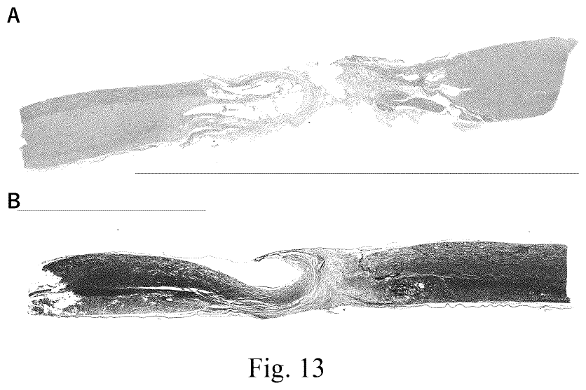

FIG. 13 each show an image of hematoxylin-eosin (HE) staining of a vertical cross-section of an excised (transplanted) site 6 weeks after excision (transplantation) of the thoracic cord in a rat into which the nerve fascicle has been transplanted immediately after the excision of the thoracic cord and a rat into which no nerve fascicle implant has been transplanted. FIG. 13A is an image of HE staining of a vertical cross-section of an excised site 6 weeks after excision of the thoracic cord in a rat into which no nerve fascicle implant has been transplanted after the excision of the thoracic cord. FIG. 13B is an image of HE staining of a vertical cross-section of a transplanted site 6 weeks after implantation in a rat into which the nerve fascicle implant has been transplanted immediately after the excision of the thoracic cord. The photograph of FIG. 13B showed that the transplantation of the nerve fascicle implant into the excised site of the thoracic cord resulted in the joining of the nerve fascicle implant to the excised site of the thoracic cord.

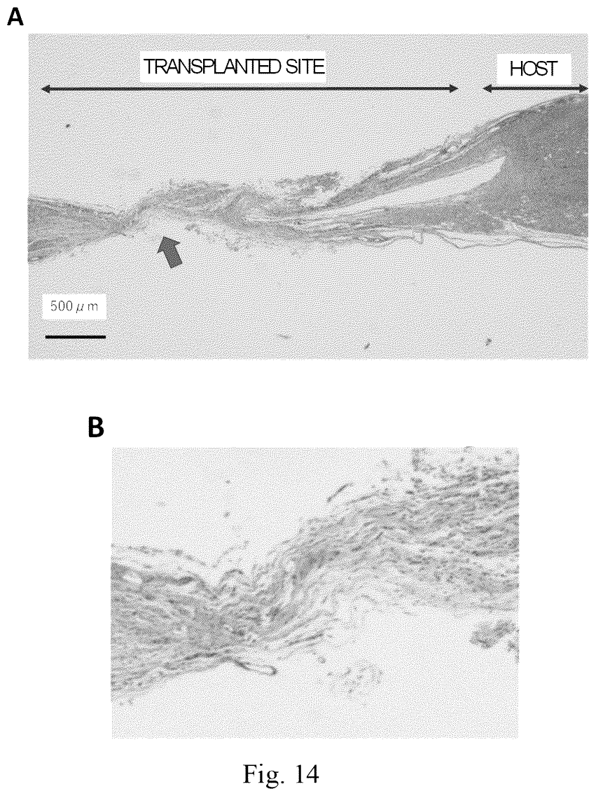

FIG. 14 each show an image of HE staining of a vertical cross-section of a transplanted site 6 weeks after excision (implantation) of the thoracic cord in a rat into which the nerve fascicle implant has been transplanted immediately after the excision of the thoracic cord. FIG. 14A is an HE stained image of the entire implanted site. FIG. 14B is an enlarged HE stained image of a joint portion (portion indicated by an arrow in FIG. 14A) between the nerve fascicle implant and the nerve portion of the excised thoracic cord of the rat (host) in the stained image in FIG. 14A. In particular, the photograph in FIG. 14B showed that the transplanted nerve fascicle implant preserved the continuity of the nerve with the cells even 6 weeks after transplantation, and was joined to the nerve portion of the excised thoracic cord of the rat (host).

These results showed that the nerve fascicle implant transplanted to the excised site of the thoracic cord was joined to the excised site of the thoracic cord, and functioned in place of the nerve portion of the thoracic cord.

### Example 9: Immunofluorescence Staining of Nerve Fascicle 1

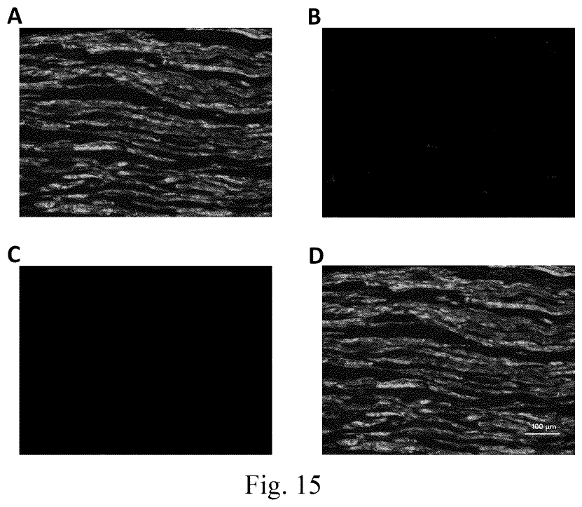

The nerve fascicle formed in Example 7 was fixed with 4% paraformaldehyde, and immunofluorescence staining was performed with p75NTR and S100 to confirm the presence or absence of expressions of p75NTR and S100. Then, the nerve fascicle was cut parallel to the extension direction of axons of neurons. Each photograph of the cut surface (vertical cross-section) of the cut nerve fascicle is shown in FIG. 15. The photographs in FIGS. 15A to 15C show the expressions of S100, p75NTR, and DAPI, respectively. The photograph of FIG. 15D is a merged photograph of FIGS. 15A to 15C. In the nerve fascicle, expressions of p75NTR and S100 along the extension direction of axons of neurons were recognized from the photographs of FIG. 15.

### Example 10: Immunofluorescence Staining of Nerve Fascicle 2

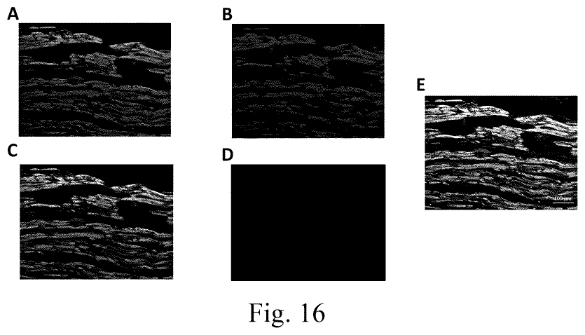

The nerve fascicle formed in Example 7 was fixed with 4% paraformaldehyde, and immunofluorescence staining was performed with HNK-1 carbohydrate, p75NTR, and MPZ to confirm the presence or absence of expressions of HNK-1 carbohydrate, p75NTR, and MPZ. Then, the nerve fascicle was cut parallel to the extension direction of axons of neurons. Each photograph of the cut surface (vertical cross-section) of the cut nerve fascicle is shown in FIG. 16. The photographs in FIGS. 16A to 16D show the expressions of HNK-1 carbohydrate, p75NTR, MPZ, and DAPI, respectively. FIG. 16E is a merged photograph of FIGS. 16A to 16D. In the nerve fascicle, expressions of HNK-1, p75NTR, and MPZ along the extension direction of axons of neurons were recognized from the photographs of FIGS. 16A to 16C. From the photograph of FIG. 16E, it was further confirmed that HNK-1, p75NTR, and MPZ were expressed in approximately the same position. MPZ is known as a marker for myelin sheath, particularly immature myelin sheath. Thus, it was suggested from the photograph of FIG. 16E that the nerve fascicle included neurons with myelin sheath and that HNK-1 and p75NTR were expressed in the myelin sheath.

### Example 11: Immunofluorescence Staining of Nerve Fascicle 3

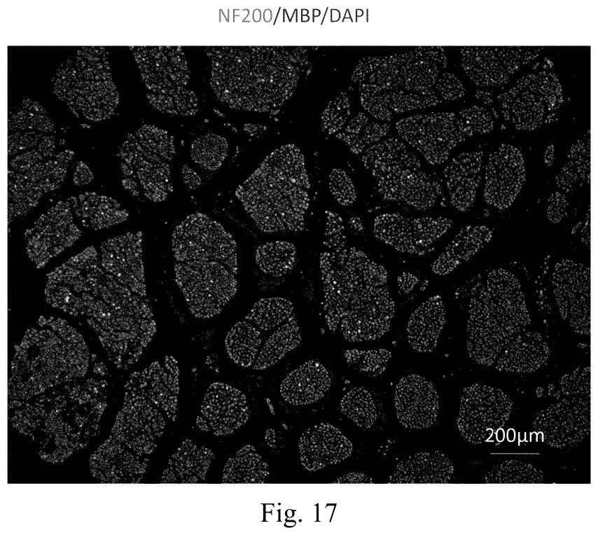

The nerve fascicle formed in Example 7 was fixed with 4% paraformaldehyde, and immunofluorescence staining was performed with NF200 and myelin basic protein to confirm the presence or absence of expressions of NF200 and myelin basic protein. Then, the nerve fascicle was cut at a right angle to the extension direction of axons of neurons. A photograph of the cut surface (cross-section) of the cut nerve fascicle is shown in FIG. 17. Expressions of NF200 and myelin basic protein around the NF200 were recognized from the photograph of FIG. 17. NF200 is known as a neuronal marker for myelinated nerves, and the myelin basic protein is known as a marker for Schwann cells. Thus, it was suggested from the photograph of FIG. 17 that the nerve fascicle included neurons with the myelin sheath of Schwann cells.

### Example 12: Immunofluorescence Staining of Nerve Fascicle 4

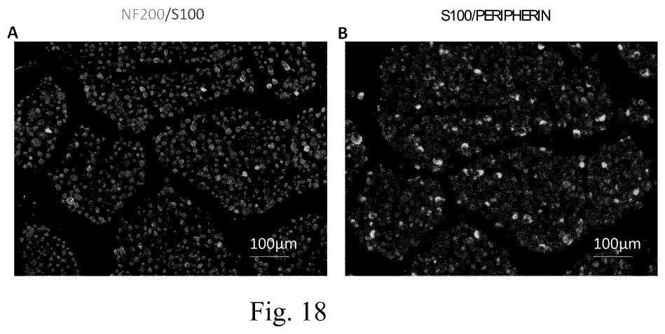

The nerve fascicle formed in Example 7 was fixed with 4% paraformaldehyde, and immunofluorescence staining was performed with NF200, S100, and peripherin to confirm the presence or absence of expressions of NF200, S100, and peripherin. Then, the nerve fascicle was cut at a right angle to the extension direction of axons of neurons. Each photograph of the cut surface (cross-section) of the cut nerve fascicle is shown in FIG. 18. The photograph of FIG. 18A shows the expressions of NF200 and S100. FIG. 18B shows the expressions of S100 and peripherin. Expressions of NF200 and S100 around the NF200 were recognized from the photograph of FIG. 18A. NF200 is known as a neuronal marker for myelinated nerves and S100 is known as a marker for Schwann cells. Thus, it was suggested from the photograph of FIG. 18A that the nerve fascicle included neurons with the myelin sheath of Schwann cells. Further, expressions of peripherin and S100 around the peripherin were recognized from the photograph of FIG. 18B. Peripherin is primarily known as a neuronal marker for the peripheral nervous system. Thus, it was suggested from FIG. 18B that the nerve fascicle included neurons with the myelin sheath of Schwann cells.

### Example 13: Immunofluorescence Staining of Nerve Fascicle 5

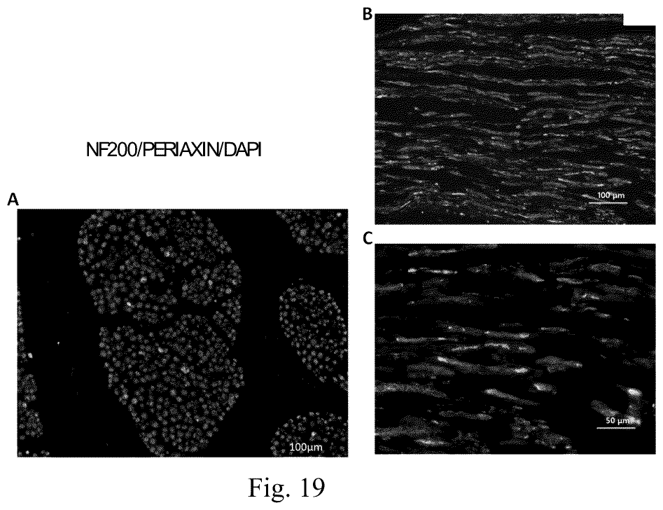

The nerve fascicle formed in Example 7 was fixed with 4% paraformaldehyde, and immunofluorescence staining was performed with NF200 and the periaxin to confirm the presence or absence of expressions of NF200 and periaxin. Then, the nerve fascicle was cut at a right angle to and parallel to the extension direction of axons of neurons. A photograph of the cut surface (cross-section) of the cut nerve fascicle is shown in FIG. 19A. Each photograph of the cut surface (vertical cross-section) of the cut nerve fascicle is shown in FIGS. 19B and 19C. Expressions of NF200 and periaxin around the NF200 were recognized from the photograph of FIG. 19A. Further, expressions of NF200 and periaxin along the extension direction of axons of neurons were recognized from the photographs of FIGS. 19B and 19C. NF200 is known as a neuronal marker for myelinated nerves and periaxin is known as a marker for Schwann cells. Thus, it was suggested from the photographs of FIGS. 19A to 19C that the nerve fascicle included neurons with the myelin sheath of Schwann cells.

### Example 14: Immunofluorescence Staining of Nerve Fascicle 6

The nerve fascicle formed in Example 7 was fixed with 4% paraformaldehyde, and immunofluorescence staining was performed with CD31 and PDGFRβ to confirm the presence or absence of expressions of CD31 and PDGFRβ. Then, the nerve fascicle was cut at a right angle to the extension direction of axons of neurons. Each photograph of the cut surface (cross-section) of the cut nerve fascicle is shown in FIGS. 20A and 20B. Expressions of CD31 and PDGFRβ around neurons (axons) of the nerve fascicle were recognized from the photographs of FIGS. 20A and 20B. CD31 is known as a marker for vascular endothelial cells, and PDGFRβ is known as a marker for vascular pericytes and fibroblasts. Thus, it was suggested from the photographs of FIGS. 20A to 20B that the nerve fascicle included a pericyte or fibroblast layer around the axons of the neurons and included a tube of vascular endothelial cells present in the layer.

### Example 15: Immunofluorescence Staining of Nerve Fascicle 7

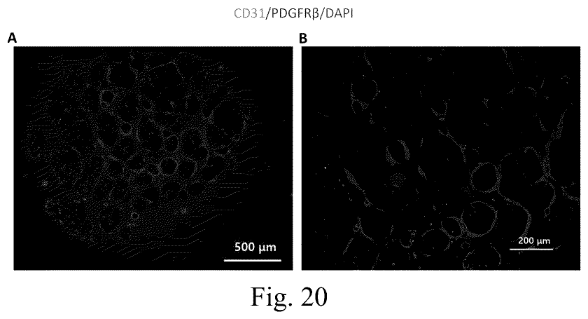

The nerve fascicle formed in Example 7 was fixed with 4% paraformaldehyde, and immunofluorescence staining was performed with CD31 and PDGFRβ to confirm the presence or absence of expressions of CD31 and PDGFRβ. Then, the nerve fascicle was cut at a right angle to the extension direction of axons of neurons. Each photograph of the cut surface (cross-section) of the cut nerve fascicle is shown in FIG. 21. FIG. 21A is a partially enlarged photograph of the cut surface (cross-section) of the nerve fascicle, and FIG. 21B is an enlarged photograph of a site where particularly strong expressions of CD31 and PDGFRβ are observed in the photograph of FIG. 21A. It was confirmed from the photographs in FIGS. 21A and 21B that CD31 was expressed in a circular form, and further, PDGFRβ was expressed to surround the circle of CD31. CD31 is known as a marker for vascular endothelial cells, and PDGFRβ is known as a marker for vascular pericytes and fibroblasts. Thus, it was suggested from the photographs of FIGS. 21A to 21B that the nerve fascicle included a pericyte or fibroblast layer around the axons of the neurons and had a tube of vascular endothelial cells present in the layer, and further, the tube of vascular endothelial cells was lined with vascular pericytes and/or fibroblasts.

### Example 16: Immunofluorescence Staining of Nerve Fascicle 8

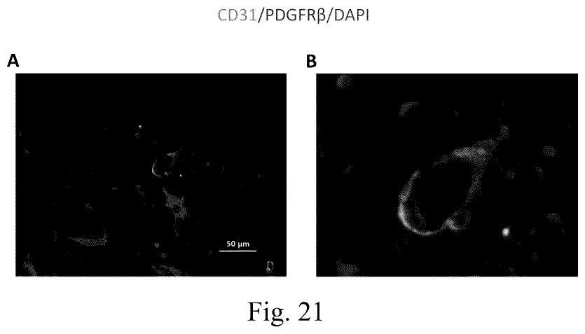

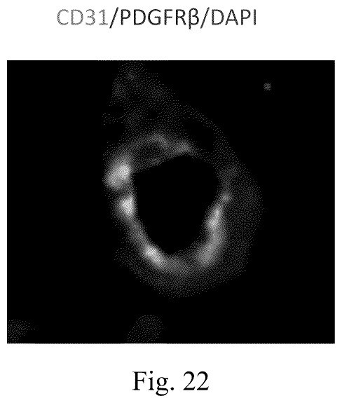

FIG. 22 shows an enlarged photograph of a site where particularly strong expressions of CD31 and PDGFRβ are observed in the cut surface (cross-section) of another nerve fascicle that has been subjected to immunofluorescence staining and cutting in a similar manner to Example 15. It was confirmed from the photograph in FIG. 22 that CD31 was expressed in a circular form, and further, PDGFRβ was expressed to surround the circle of CD31. Thus, it was suggested from the photograph of FIG. 22 that the nerve fascicle included a pericyte or fibroblast layer around the axons of the neurons and included a tube of vascular endothelial cells present in the layer, and further, the tube of vascular endothelial cells was lined with vascular pericytes and/or fibroblasts.

### Example 17: Transplantation of Nerve Fascicle 2 (Transplantation into Rat Sciatic Nerve)

The nerve fascicle implant formed in Example 7 was transplanted into the sciatic nerve in rats according to the following method.

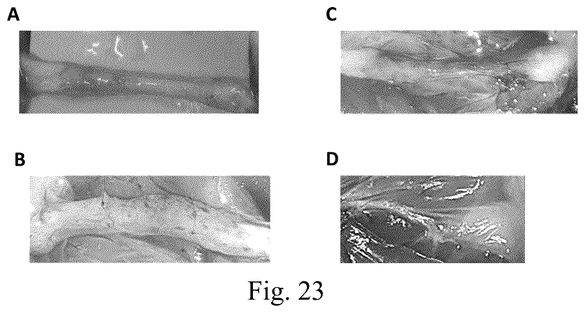

Rats (nude rats; F344/NJcl-rnu/mu, 15-week-old, male, available from CLEA Japan, Inc.) were subjected to general anesthesia by intraperitoneal administration of three types of mixed anesthetic agents (a mixture of medetomidine hydrochloride (0.15 mg/0.15 ml/kg), midazolam (2 mg/0.4 ml/kg), butorphanol tartrate (2.5 mg/0.5 ml/kg), and physiological saline (1.45 ml/kg)), and a part of the sciatic nerve was excised. Each of the two ends of the nerve fascicle implant formed in Example 6 (about 1 cm in length, about 1 mm in diameter) was sutured to each of the two nerve stumps after sciatic nerve excision with 3 stitches of surgical sutures (10-0 nylon), and the nerve fascicle implant was transplanted into the excised sciatic nerve portion. A rat in which the distal and proximal ends of the excised nerve had been inverted and transplanted according to the similar method described above (autologous nerve-transplanted rat) as well as a rat transplanted with an artificial nerve (Nerbridge (registered trademark), manufactured by TOYOBO CO., LTD.) were prepared. Further, a rat in which the sciatic nerve had been incised (sciatic nerve-incised rat) was prepared. Photographs of the transplanted sites and the incised site in the rats 12 weeks after transplantation are shown in FIG. 23 (A: autologous nerve-transplanted rat; B: nerve fascicle implant-transplanted rat; C: artificial nerve-transplanted rat; D: sciatic nerve-incised rat). The photographs of FIG. 23 showed that the transplanted autologous nerve, the transplanted nerve fascicle implant, and the transplanted artificial nerve were joined to the sciatic nerve of the autologous nerve-transplanted rat, the sciatic nerve of the nerve fascicle implant-transplanted rat, and the sciatic nerve of the artificial nerve-transplanted rat, respectively. Further, in the case where the nerve fascicle implant was transplanted (FIG. 23B), the nerve fascicle was significantly covered with fibroblasts as compared with the case where the autologous nerve was transplanted or the case where the artificial nerve was transplanted (FIGS. 23A and 23C).

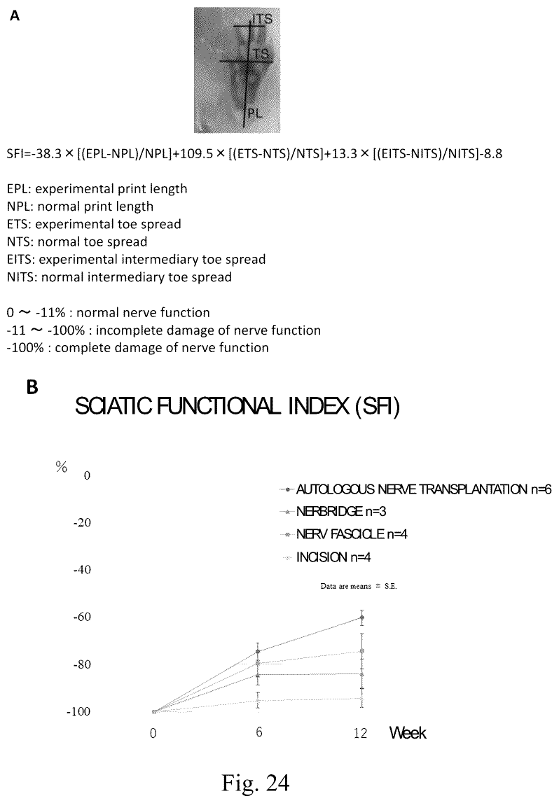

In addition, 6 autologous nerve-transplanted rats, 3 nerve fascicle implant-transplanted rats, 4 artificial nerve-transplanted rats, and 4 sciatic nerve-incised rats were prepared, and changes in the sciatic functional index (SFI) after transplantation (or after incision) in each of the rats were observed. Note that the sciatic functional index is described in the reference “Bain et al., Plast Reconstr Surg 83: 129-139 (1989). The sciatic functional index was calculated from the footprints obtained by allowing each rat with inked volar pads to walk, based on the mathematical formula shown in FIG. 24A. FIG. 24B shows temporal changes in the sciatic functional index in each of the rats. FIG. 24B showed that the sciatic functional index 6 weeks after transplantation increased in the autologous nerve-transplanted rats, nerve fascicle implant-transplanted rats, and artificial nerve-transplanted rats, as compared with the sciatic functional index immediately after transplantation. It was also shown that, in the autologous nerve-transplanted rats and the nerve fascicle implant-transplanted rats, the sciatic functional index 12 weeks after transplantation further increased as compared with the sciatic functional index 6 weeks after transplantation.

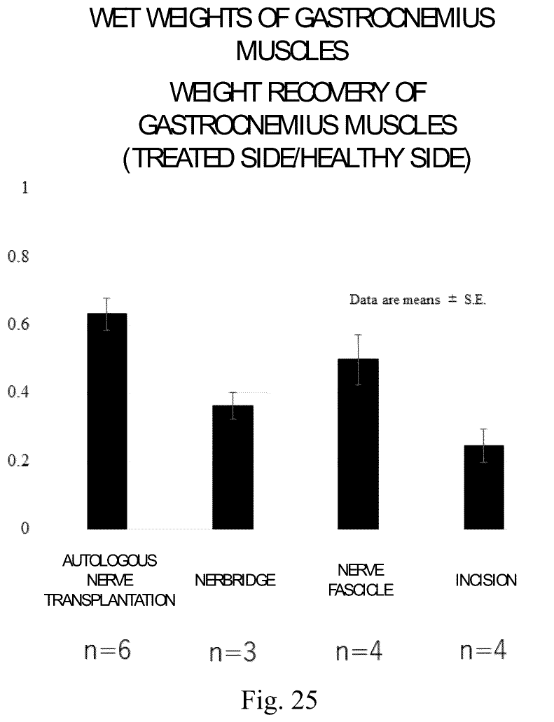

Concerning each of the rats after calculation of the sciatic functional index, the gastrocnemius muscle at the distal end of the transplanted site was excised in the 12th week after transplantation (or after incision), and a ratio of the wet weight after excision to the wet weight of the gastrocnemius muscle in the rat with non-excised sciatic nerve was used for measurement. The measurement results are shown in FIG. 25. FIG. 25 showed that the weight of the gastrocnemius muscle in the autologous nerve-transplanted rats, nerve fascicle implant-transplanted rats, and artificial nerve-transplanted rats was larger than the weight of the gastrocnemius muscle in the sciatic nerve-incised rats; and the autologous nerve, the nerve fascicle implant, and the artificial nerve each served as the rat sciatic nerve.

### Example 18: Immunostaining of Nerve Fascicle after Transplantation into Rat Sciatic Nerve

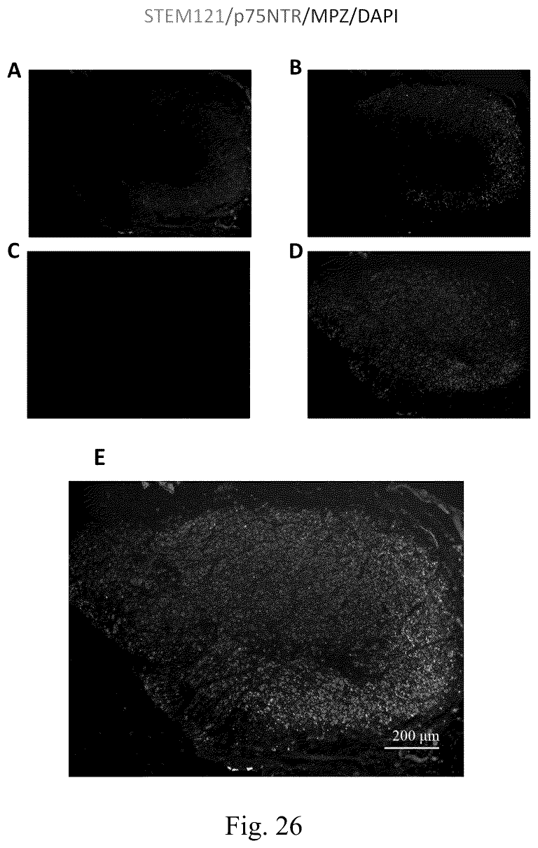

The nerve fascicle implant transplanted into the rat sciatic nerve in Example 17 was excised 12 weeks after transplantation. The excised nerve fascicle implant was fixed with 4% paraformaldehyde, and immunofluorescence staining was performed with STEM121, p75NTR, and MPZ to confirm the presence or absence of expressions of STEM121, p75NTR, and MPZ. Then, the nerve fascicle implant was cut at a right angle to the extension direction of axons of neurons. Each photograph of the cut surface (cross-section) of the cut nerve fascicle implant is shown in FIG. 26. The photographs in FIGS. 26A to 26D show the expressions of STEM121, p75NTR, DAPI, and MPZ, respectively. FIG. 26E is a merged photograph of FIGS. 26A to 26D. It was recognized from the photographs of FIGS. 26A, 26B, and 26D that STEM121, p75NTR, and MPZ were expressed at approximately the same position. STEM121 shows neurons in the nerve fascicle implant. Consequently, this suggested that p75NTR and MPZ were expressed in the nerve fascicle in the nerve fascicle implant, particularly neurons.

### INDUSTRIAL APPLICABILITY

According to the present invention, it is possible to efficiently extend and enlarge axons of neural cells. As a result, a nerve fascicle having axons with a length and diameter sufficient for transplantation can be efficiently formed, allowing for efficient provision of a nerve fascicle implant necessary for nerve transplantation.

### REFERENCE SIGNS LIST

1 Nerve fascicle forming device
2 Dimethylpolysiloxane substrate
3 Recess
4 Recess (distal recess)
5 Channel portion

1. A method of producing a nerve fascicle, the method comprising cultivating a neural cell-containing cell population in the presence of glial cells and at least one type of feeder cells selected from the group consisting of vascular component cells, perivascular cells, and oligodendrocytes to extend axons of neural cells.

2. The method according to claim 1, wherein the feeder cells include at least one type of cells selected from the group consisting of pericytes, vascular endothelial cells, fibroblasts, smooth muscle cells, and oligodendrocytes.

3. The method according to claim 1 or 2, wherein the feeder cells include cells secreting at least one type of growth factor selected from the group consisting of VEGF, NGF, BDNF, FGF-2, NGFB, and EGF.

4. The method according to any one of claims 1 to 3, wherein the glial cells include oligodendrocytes.

5. The method according to any one of claims 1 to 4, wherein the nerve fascicle includes a myelin sheath containing oligodendrocytes.

6. The method according to any one of claims 1 to 5, comprising:
(a) preparing a substrate including at least one recess and a channel portion connected to the recess, the channel portion being covered with the feeder cells;
(b) adding the neural cell-containing cell population and the glial cells to the recess; and
(c) cultivating the neural cell-containing cell population and the glial cells to extend the axons of the neural cells along the channel portion.

7. The method according to any one of claims 1 to 6, comprising:
(a) preparing a substrate including two recesses and a channel portion connecting the two recesses, the channel portion being covered with the feeder cells;
(b) adding the neural cell-containing cell population and the glial cells to each of the two recesses; and
(c) cultivating the neural cell-containing cell population and the glial cells to extend the axons of the neural cells along the channel portion.

8. The method according to claim 6 or 7, wherein, in the step (a), the channel portion is covered with fibroblasts before covered with the feeder cells.

9. The method according to any one of claims 1 to 8, wherein the neural cell-containing cell population further includes endothelial cells.

10. The method according to claim 9, wherein the endothelial cells included in the neural cell-containing cell population are blood vessel-derived endothelial cells.

11. The method according to claim 10, wherein the blood vessel-derived endothelial cells are blood vessel-derived endothelial cells in at least one tissue selected from the group consisting of dental pulp, a gingiva, a subcutaneous tissue, a coelomic artery, a coelomic vein, and an umbilical cord.

12. The method according to any one of claims 9 to 11, further comprising, in the step (c), forming a tube derived from the endothelial cells in the neural cell-containing cell population.

13. The method according to any one of claims 9 to 12, wherein the neural cells and the endothelial cells are derived from an identical individual.

14. The method according to any one of claims 9 to 13, wherein the neural cell-containing cell population further includes a biocompatible material, and the neural cells and the endothelial cells are layered on a surface of a different biocompatible material, respectively.

15. The method according to claim 14, wherein the biocompatible material includes a collagen.

16. The method according to claim 14 or 15, wherein the biocompatible material includes collagen beads.

17. The method according to any one of claims 6 to 16, wherein the channel portion has a length of 3 mm or greater.

18. A nerve fascicle produced by the method described in any one of claims 1 to 17.

19. A method of producing an implant comprising covering a nerve fascicle with a sheet of a biocompatible material, the nerve fascicle being produced by the method described in any one of claims 1 to 17.

20. The method according to claim 19, wherein the sheet includes fibroblasts.

21. The method according to claim 19 or 20, wherein the implant is a nerve regeneration implant.

22. An implant produced by the method described in any one of claims 19 to 21.

23. A method of extending axons of neural cells, the method comprising cultivating neural cells in the presence of glial cells and feeder cells including at least one type of cells selected from the group consisting of vascular component cells, perivascular cells, and oligodendrocytes.

24. The method according to claim 23, wherein the feeder cells include at least one type of cells selected from the group consisting of pericytes, endothelial cells, fibroblasts, smooth muscle cells, and oligodendrocytes.

25. The method according to claim 23 or 24, wherein the glial cells include oligodendrocytes.

26. A nerve fascicle comprising:
neural cells with axons extended; and
a tube of endothelial cells present along the axons,
wherein
the nerve fascicle includes at least one type of cells of HNK-1 carbohydrate-expressing cells or p75NTR-expressing cells, and
the axons have a myelin sheath containing oligodendrocytes.

27. The nerve fascicle according to claim 26, comprising:
HNK-1 carbohydrate-expressing cells; and
p75NTR-expressing cells.

28. The nerve fascicle according to claims 26 and 27, comprising at least one type of cells selected from the group consisting of NS200-expressing cells, peripherin-expressing cells, myelin basic protein-expressing cells, S100-expressing cells, MPZ-expressing cells, periaxin-expressing cells, CD31-expressing cells, and PDGFRβ-expressing cells.

29. The nerve fascicle according to any one of claims 26 to 28, comprising: NS200-expressing cells;
peripherin-expressing cells;
myelin basic protein-expressing cells;
S100-expressing cells;
MPZ-expressing cells;
periaxin-expressing cells;
CD31-expressing cells; and
PDGFRβ-expressing cells.

30. The nerve fascicle according to any one of claims 26 to 29, comprising a cell layer including at least one cell selected from fibroblasts or pericytes that covers, along the axons of the neural cells, at least some of the axons, wherein the tube of endothelial cells is present in the cell layer.

31. The nerve fascicle according to any one of claims 26 to 30, wherein the tube of endothelial cells further includes pericytes.

32. An implant comprising the nerve fascicle described in any one of claims 26 to 31.

33. The implant according to claim 32, which is a nerve regeneration implant.
